SEQUENCE LISTING (10 sequences)

SEQ ID NO: 1 - DNA, 20 bp, synthetic construct
  source 1..20: mol_type = other DNA
acagagcgggacaggtgggt

SEQ ID NO: 2 - DNA, 20 bp, synthetic construct
  source 1..20: mol_type = other DNA
cctcgggaggccccggttta

SEQ ID NO: 3 - DNA, 18 bp, synthetic construct
  source 1..18: mol_type = other DNA
ccctcactcaggtcgcct

SEQ ID NO: 4 - DNA, 18 bp, synthetic construct
  source 1..18: mol_type = other DNA
cgtgcgaggagccaatca

SEQ ID NO: 5 - DNA, 20 bp, synthetic construct
  source 1..20: mol_type = other DNA
acatctcccagatcccacag

SEQ ID NO: 6 - DNA, 20 bp, synthetic construct
  source 1..20: mol_type = other DNA
gggcatttggttgtctcagt

SEQ ID NO: 7 - DNA, 23 bp, synthetic construct
  source 1..23: mol_type = other DNA
acaccgctgttgttttccagagt

SEQ ID NO: 8 - DNA, 24 bp, synthetic construct
  source 1..24: mol_type = other DNA
ccttcagctccccgtgaaatcaaa

SEQ ID NO: 9 - DNA, 20 bp, synthetic construct
  source 1..20: mol_type = other DNA
agagcggctgacaatagagg

SEQ ID NO: 10 - DNA, 18 bp, synthetic construct
  source 1..18: mol_type = other DNA
gtgcgctcaccagctgac

(19) United States
(12) Patent Application Publication
(10) Pub. No.: US 2024/0245702 A1
(43) Pub. Date: Jul. 25, 2024

(54) Inhibition of Lysine Demethylase 1 (Lsd1) Induces Differentiation of Hair Cells

(71) Applicant: Massachusetts Eye and Ear Infirmary, Boston, MA (US)

(72) Inventors: Albert Edge, Brookline, MA (US); Niliksha Gunewardene, Boston, MA (US)

(21) Appl. No.: 18/491,200

(22) Filed: Oct. 20, 2023

Related U.S. Application Data
(63) Continuation of application No. 17/296,764, filed on May 25, 2021, now abandoned.
(60) Provisional application No. 62/773,965, filed on Nov. 30, 2018.

(51) Int. Cl. A61K 31/55; A61K 31/137; A61K 31/506; A61K 45/06

(52) U.S. Cl. CPC A61K 31/55; A61K 31/137; A61K 31/506; A61K 45/06

(57) ABSTRACT

Methods for the generation of sensorineural hair cells, and more particularly to the use of epigenetic modulation of Atoh1 expression using a combination of Histone Lysine Demethylase (KDM) inhibitors and Wnt activators to generate sensorineural hair cells.

### CLAIM OF PRIORITY

This application is a continuation of U.S. patent application Ser. No. 17/296,764, filed May 25, 2021, which is a national stage entry of PCT/US2019/063418, filed Nov. 26, 2019, which claims the benefit of U.S. Provisional Patent Application Ser. No. 62/773,965, filed Nov. 30, 2018. The entire contents of the foregoing are hereby incorporated by reference.

### FEDERALLY SPONSORED RESEARCH OR DEVELOPMENT

This invention was made with Government support under Grant No. DC14089 awarded by the National Institutes of Health. The Government has certain rights in the invention.

### SEQUENCE LISTING

This application contains a Sequence Listing that has been submitted electronically as an XML file named “00633-0262002_SL ST26.XML.” The XML file, created on Oct. 20, 2023, is 9,852 bytes in size. The material in the XML file is hereby incorporated by reference in its entirety

### TECHNICAL FIELD

This invention relates to the generation of sensorineural hair cells, and more particularly to the use of epigenetic modulation of Atoh1 expression using a combination of Histone Lysine Demethylase (KDM) inhibitors and Wnt activators to generate sensorineural hair cells.

### BACKGROUND

There are six distinct sensory organs in the mammalian inner ear: the three cristae of the semicircular canals, the two maculae of the saccule and utricle, and the organ of Corti of the cochlea. The organ of Corti is the organ of hearing. The receptor cell for hearing is the hair cell of the cochlea (referred to herein as a hair cell, a sensory hair cell, or a sensorineural hair cell). Hair cells are limited in number and do not regenerate in mammals; damage or death of these cells leads to hearing loss (Edge and Chen, Curr. Opin. Neurobiol., 18:377-382 (2008)).

### SUMMARY

Epigenetic silencing of transcription factors crucial for cell fate determination is a possible cause of the loss of regenerative capacity in adult sensory systems. Hair cells, the receptor cells for sound, are a vulnerable part of the auditory pathway, leading to deafness in the absence of regeneration. BHLH transcription factor, Atoh1, is required for embryonic hair cell differentiation, and its overexpression in the new born cochlea is sufficient to transdifferentiate other sensory epithelial cells to hair cells, but expression in the adult cochlea is downregulated. Atoh1 is downstream of Wnt signaling, and while Wnt stimulation alone is not sufficient to restore hair cells in the adult cochlea, we asked whether inhibition of epigenetic modifier, Lsd1, in combination with Wnt signaling activated Wnt downstream targets, including Atoh1. Lsd1 bound directly to the Atoh1 locus, and CRISPR-Cas9 mediated delivery of Lsd1 to the Atoh1 locus decreased Atoh1 expression. Inhibition or genetic silencing of Lsd1 increased H3K4me2 marks on the Atoh1 enhancer and allowed the upregulation of Atoh1 mRNA in the mouse cochlea and stimulated the differentiation of supporting cells to hair cells. Since the effect on hair cell differentiation was only seen when Lsd1 inhibition was combined with Wnt activation, we hypothesized that Lsd1 maintained Wnt targets in a silenced/poised state and that Lsd1 inhibition made the supporting cells responsive to Wnt. This inhibition increased Atoh1 expression in the adult, where Atoh1 is normally silenced and thereby contributed to the differentiation of new hair cells after damage. This mechanism for activation of transcription factors that become silenced in the postnatal animal thus provides a new avenue for the restoration of hearing.

Thus, described herein are methods for treating sensorineural hearing loss associated with loss of auditory hair cells or balance loss associated with a loss of vestibular hair cells in a subject. The methods include administering to the subject, preferably to the ear, e.g., to the inner ear, of the subject: (i) a pharmaceutical composition comprising a Histone Lysine Demethylase (KDM) inhibitor; and (ii) a pharmaceutical composition comprising a Wnt agonist.

Also described herein is the use of a Histone Lysine Demethylase (KDM) inhibitor and a Wnt agonist for the treatment of sensorineural hearing loss associated with loss of auditory hair cells in a subject. In some embodiments, the KDM inhibitor and Wnt agonist are formulated for administering to the ear of the subject, preferably to the inner ear of the subject.

In some embodiments, the KDM inhibitor is selected from the group consisting of tranylcypromine (trans-2-phenylcyclopropyl-1-amine, trans-2-PCPA, TCP) and analogs thereof, e.g., with one or more substitutions, e.g., at the benzene ring or at the amine position ((e.g., ORY-1001 (rel-N1-[(1R,2S)-2-phenylcyclopropyl]-1,4-cyclohexanediamine, dihydrochloride): S2101 ((1R,2S)-rel-2-[3,5-Difluoro-2-(phenylmethoxy)phenyl]cycloprpanamine hydrochloride); or GSK-LSD1 (rel-N-[(1R,2S)-2-Phenylcyclopropyl]-4-Piperidinamine hydrochloride)): 2,4-pyridinedicarboxylic acid (2,4-PDCA): 5-Carboxy-8-hydroxy quinoline (IOX1) and n-octyl ester thereof: Pargyline (N-Methyl-N-propargylbenzylamine) or Pargyline hydrochloride (N-Methyl-N-propargylbenzylamine hydrochloride); and C12 ((E)-N′-(1-(5-chloro-2-hydroxyphenyl)ethylidene)-3-(morpholinosulfonyl)benzohydrazide).

In some embodiments, the Wnt agonist is set forth in Table A.

In some embodiments, the Wnt agonist is a GSK3β antagonist.

In some embodiments, the subject is a mammal, e.g., a human, e.g., who is at least 3 months of age.

Unless otherwise defined, all technical and scientific terms used herein have the same meaning as commonly understood by one of ordinary skill in the art to which this invention belongs. Methods and materials are described herein for use in the present invention: other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1H: Pharmacological and genetic inhibition of Lsd1 inhibition potentiates hair cell differentiation (A) The effect of LSD1 inhibition on proliferation was analyzed using Lgr5-nGFP mice. The Lsd1 inhibitor, pargyline (PA) combined with CHIR99021 enhanced LCP proliferation (p<0.05*), but the effect was less than that of the HDAC inhibitor, valproic acid (VP: p<0.01**). (B) To assess the effect of Lsd1 inhibitors on hair cell differentiation, cells from Atoh1-nGFP mice were expanded for 10 days (referred to as DO) and then differentiated under the conditions described in Methods with the addition of pargyline for an additional 10 days (D10). Pargyline increased the percentage of Atoh1-nGFP cells (quantified using FACS: p<0.05*). (C and D) Treatment with pargyline increased the percentage of Atoh1 and Myo7a (qRT-PCR: p<0.05*) relative to the untreated. (E) To further probe the effect of Lsd1 inhibitors on hair cell differentiation, organoids from Sox2-Cre; Atoh1-nGFP:Lsd1^{fl/+} mice were expanded for 10 days and treated with tamoxifen at the start of the differentiation phase (D0), to induce heterozygous Lsd1 knockout (LSD1 KO). An increased percentage of hair cells was revealed by FACS analysis for Atoh1-nGFP (p<0.05*). (F) While Atoh1 expression was not changed significantly, Myo7a upregulation was observed. (G and H) The organoids treated with the Lsd1 inhibitor combination were nearly uniformly positive for Atoh1 (G) and Myo7a (H). The Myo7a cells also had actin-rich protrusions comprising several individual stereocilia (H). Scale bars=50 μM: error bars represent mean±SEM. *** p<0.001: *p<0.05: n≥3 for all experiments.

FIGS. 2A-2D: Lsd1 inhibition alters the gene expression profile of cochlear progenitors and activates expression of hair cell genes (A) Heat map of the normalized counts for all the significantly expressed genes reveals that Lsd1 inhibitor treatment significantly alters the gene profile of LCPs. (B) Volcano plot of the differentially expressed genes in the pargyline-treated LCPs, relative to the untreated. (C) Gene set enrichment analysis was used to examine enrichment in gene ontology terms in the treated samples. Hair cell genes (GO:0060117) were significantly enriched (enrichment score-0.6, padj<0.1). (D) Fold changes of the most differentially expressed genes in the pargyline-treated samples.

FIGS. 3A-3D: Lsd1 inhibitor potentiates the Wnt/β-catenin pathway (A) Western blot for active β-catenin in the Lsd-inhibitor and untreated samples after 4 days of differentiation show increased levels in the pargyline and CHIR99021 (PACH) and pargyline, CHIR99021 and LY411575 (PALYCH)-treated samples, relative to the untreated control. (B) No significant difference was observed in Wnt activity in cells treated with CHIR99021 relative to pargyline and CHIR99021, as measured by a luciferase assay for Tcf/Lef. Error bars represent mean±SEM: n≥3. (C) Heat map of the normalized counts of the Wnt target genes that are differentially expressed at the start of differentiation (DO) and after 10 days of differentiation (D10) with and without pargyline (PA). The heat map depicts significant clustering of the Lsd1 inhibitor treated relative to the untreated samples. (D) Normalized counts of the top 15 differentially expressed Wnt-target genes in differentiated LCPs (p<0.05). For the Western blot and luciferase assay experiments, n≥3.

FIGS. 4A-4E: Hair cell differentiation is suppressed upon targeting a nuclease deficient dCas-9 Lsd1 to the promoter or enhancer regions of the Atoh1 gene (A) The expression of Kdm1a (Lsd1) is maintained in the cochlea from postnatal day 0 to adult (P28). (B) The mRNA levels of Atoh1 significantly declined from PO to P28 (p<0.05*, 0.001***). (C) Using ChIP-qPCR, we observed enrichment of Lsd1 at the promoter and enhancer regions of the Atoh1 gene in LCP-derived hair cells. (n≥3). (D) Targeting the promoter and enhancer regions of the Atoh1 gene using dCas9-Lsd1 led to a loss of Atoh1 and Myo7a expression in the transduced cells (p<0.05*. 0.01**). (E) Schematic of the mouse Atoh1 locus, depicting the regions analyzed, distal promoter (−1108 to −1023), proximal promoter (−332 to −128), coding region (1176-1294), enhancer 1 (4284 to 4395) and enhancer 2 (5366-5457). Error bars represent mean±SEM. *** p<0.001: * p<0.05.

FIGS. 5A-5E: Chromatin immunoprecipitation reveals that Atoh1 upregulation is concurrent with the accumulation of H3K4me and H3K4me2 at the promoter and enhancer regions of the Atoh1 locus. (A) Timeline used for the differentiation of LCPIs into hair cells. The cells were harvested at the start of differentiation (D0) and then at the end of differentiation with and without pargyline (PA). (B) A significant increase in the level of H3K4me2 was observed at the proximal promoter (region 2) at the end of differentiation (D10). This level was further increased by pargyline treatment (p<0.05*, p<0.01**). (C) The level of H3K4me was significantly increased at the enhancer after pargyline treatment (5366 to 5457). (D) No significant changes in the level of H3K9me2 was observed in the LCPs after treatment with pargyline. (E) The epigenetic marker expression was analyzed at several sites in the Atoh1 gene, distal promoter (−1129 to −1044), proximal promoter (−353 to −149), coding region (1155-1273), enhancer 1 (4263 to 4374) and enhancer 2 (5345-5436). All values are mean±SEM. * p<0.05, ** P<0.01.

FIGS. 6A-6E: Treatment of cochlear explants with the Lsd1 inhibitor induced increased hair cell numbers and supporting cell proliferation (A and B) Explants obtained from P2-3 Atoh1-nGFP mice were treated with 0.1% DMSO (Ctl) or pargyline and CHIR99021 (PACH). Increased outer hair cell (OHC) numbers were observed in the explants treated with pargyline and CHIR99021, when compared to untreated control (p<0.001***) Conversely, no significant difference was observed in the number of inner hair cells (IHC). (C) To assess the effect of Lsd1 inhibitors on supporting cell proliferation, cochlear explants were treated and stained for EdU. (D) Orthogonal view of a pargyline and CHIR99021-treated sample showed incorporation of EdU in the Sox2-positive supporting cells. (E) The untreated (Ctl) had significantly reduced numbers of EdU-positive cells, when compared to the treatment with pargyline and CHIR99021 (n=4). Note Myo7a labeling (red) of the hair cells. Sox2 labeling (green) of supporting cells, and EdU labeling (grey) of the supporting cells. Scale bars=50 μM.

FIGS. 7A-7G: Partial hearing recovery and transdifferentiation of supporting cells into hair cells after pargyline and CHIR treatment in the deaf adult cochlea (A) The animals were administered tamoxifen at P21 and their pre-ABRs measured. At P28, the animals were exposed to 116 dB SPL noise and the next day, cochlear function was tested to confirm deafness. The drug was then placed on the round window membrane. Cochlear function was tested again 1 week and 1 month-post treatment. (B) A decrease in ABR thresholds in the pargyline and CHIR99021-treated as compared to the untreated ears was apparent at 5.66 kHz (p<0).05*) from ABR threshold recordings made at seven frequencies from 5.66 to 45.25 kHz with the following time course: before noise exposure (pre-noise, open circles). 1 day-after noise exposure (postnoise, closed circles). 1 week after drug treatment (1-week, open squares) and 1 month after treatment (1 month, closed squared) (n=5 in each group). When no response was observed at 80 dB (maximum acoustic output of the system), the threshold was designated as 85 dB SPL. (C) No changes in DPOAEs were observed after treatment. Error bars show SD. (D) Cells positive for both the Sox2 lineage (GFP) and myosin VIIa (blue) were observed in the outer hair cell region of deafened mice carrying the Sox2-CreER and mT/mG genes. 1 month after pargyline and CHIR99021 treatment. These confocal x-y projection images of pargyline and CHIR99021-treated ears from Sox2-CreER; mT/mG double-transgenic mice are in the 5.66-8 kHz area of the cochlea. (E) Confocal x-z view of hair cells with their original (red) bundles (white arrowheads) adjacent to cells with new (green) bundles (yellow arrowheads) derived from Sox2-positive cells. (F) High power x-y view of hair cells with their original (red) bundles (white arrowheads) adjacent to cells with new (green) bundles (yellow arrowheads) derived from Sox2-positive cells. (G) Confocal x-y projection images of the untreated ears in the 5.66-8 kHz area.

FIGS. 8A-8D: Changes in expression of Wnt target genes after noise damage and treatment with pargyline and CHIR99021 (A) qRT-PCR analysis for Wnt and Notch genes was performed 3 days after drug treatment of noise-damaged animals. (B) The level of Axin2 was reduced post-noise exposure. Treatment with CHIR99021 (CH) or pargyline and CHIR99021 (PACH), partially reversed this effect. (C) Hes1 expression was significantly elevated following noise exposure. CHIR99021 or pargyline and CHIR99021 treatment reduced the level of Hes1. (D) Atoh1 expression was decreased after noise-damage. Treatment with CHIR99021 or pargyline and CHIR99021, resulted in an increase in Atoh1. The effect was most significant with the combination of pargyline and CHIR99021 (p<0.05*). mRNA levels were calculated relative to a pre-noise control (n≥6 animals per group and 4 experimental repeats).

FIGS. 9A-9D: Treatment of LCPs with Lsd1 inhibitors for their effect on proliferation (A and B) Schematic of the protocol used to differentiate LCPs towards a hair cell lineage. (C) Screening of Lsd1 inhibitors for their effect on LCP proliferation. (D) Screening of Lsd1 inhibitors for their effect on hair cell differentiation.

FIGS. 10A-10B: Assessment of variability and clustering of samples of LCPs submitted to RNA sequencing (A) Pearson correlation plot depicting the variability between samples, demonstrates clear segregation of samples by the timing of differentiation and treatment (0.98-0.99 correlation coefficient). (B) The PCA plot reveals that timing of differentiation is responsible for 62% of the variation between samples, while treatment accounts for 20% of the variation.

FIGS. 11A-11B: Analysis of LSD1 interaction with the Atoh1 locus (A) ChIP-Seq data depict LSD1 enrichment on the Atoh1 locus in embryonic stem cells and neural stem cells (Wang et al., 2016: Whyte et al., 2012). The density of LSD1 signals is higher in promoter compared to enhancer regions, while, in neural stem cells, the signals at the promoter and enhancer regions are comparable. (B) Schematic of the regions analyzed for changes in epigenetic marks after Lsd1 inhibitor treatment.

### DETAILED DESCRIPTION

A major cause of deafness is the irreversible loss of the sensory hair cells that are responsible for transducing sound into an electrical signal that can be transmitted to the brain. Although hair cells do not regenerate spontaneously (Fujioka et al., 2015: Groves, 2010), recent studies have shown that in the newborn cochlea upon damage, hair cells are regenerated from supporting cells (Bramhall et al., 2014; Cox et al., 2014). The spontaneous regeneration in these animals was dependent on both Wnt and Notch signaling, two pathways that are critical to the development of hair cells in the embryo (Bramhall et al., 2014: Hu et al., 2007). One explanation for the lack of a continued regenerative response as the animal ages is that key signaling pathways are downregulated after birth. A decreased response to damage with age appears to correlate with a decreased response to Wnt (Shi et al., 2013). bHLH transcription factor Atoh1 is a downstream targets of Wnt signaling (Shi et al., 2010) that is essential for hair cell development and shows decreasing levels of expression after birth (Cai et al., 2013; Chen et al., 2002: Chen and Segil, 1999: Chonko et al., 2013). Wnt pathway ablation by deletion of β-catenin abolishes hair cell development (Shi et al., 2014) and a similar phenotype is seen after deletion of Atoh1 (Bermingham et al., 1999). Downstream targets of Atoh1 are also downregulated as Atoh1 expression begins to fall in late development (Cai et al., 2015) and may be absent in the adult. Genetically stabilizing β-catenin or pharmacologically activating the Wnt pathway induces hair cells in the new born cochlea (Chai et al., 2012: Geng et al., 2016: Hu et al., 2016; Jacques et al., 2012; Shi et al., 2010; Shi et al., 2013: Shi et al., 2014; Shi et al., 2012). Thus, both Atoh1 and its downstream targets can be upregulated in the first postnatal weeks. However, Atoh1 is silenced in the adult and Wnt activation is insufficient to induce expression of the gene (Shi et al., 2013). The decreased ability of these proneural transcription factors to be activated was thought to account for the loss of regenerative capacity of the adult cochlea (Samarajeewa et al., 2018: Shi et al., 2013).

The effect of Wnt signaling on hair cell differentiation decreased with increasing postnatal days, such that the stimulation of Wnt signaling showed a dramatically decreased effect by P4-P7 (Samarajeewa et al., 2018: Shi et al., 2013). We have recently shown that the Wnt pathway remained active in the adult cochlea but that there was a decline in the number of genes that remained responsive to Wnt pathway activation (Samarajeewa et al., 2018). We also showed that this loss of activity was due to diminished activation of downstream targets rather than a loss of the components or activity of the Wnt pathway itself (Samarajeewa et al., 2018; Shi et al., 2013).

DNA methylation and covalent modifications of histones influence cell differentiation through their effects on transcription (Hanna et al., 2010; Jaenisch and Bird, 2003). Epigenetic changes also contribute to a decline in regenerative capacity (Hanna et al., 2010; Jaenisch and Bird, 2003: Jorstad et al., 2017: Ocampo et al., 2016; Park et al., 2016). Considerable evidence has revealed that Atoh1 is a bivalent gene, defined by the simultaneous presence of active and repressive epigenetic marks (Azuara et al., 2006). Here, we hypothesized that epigenetic modifications may be responsible for the decline in regenerative capacity in the adult cochlea. Target genes necessary for hair cell differentiation may exist in a heterochromatin state, inaccessible to transcription factor networks. We hypothesized that repressive modifiers may be responsible for inhibiting genes responsible for hair cell differentiation, such as Atoh1, and that the block to regeneration in the adult was due to the resistance of bivalent proneural targets to Wnt activation.

The lysine-specific demethylase 1 Lsd1 is an inactivator of neural progenitor genes. It was initially identified as a component of the HDAC-containing, Co-REST transcriptional repressor complex (Ballas et al., 2001). Lsd1 was later identified as a histone demethylase, specific for removing methyl groups from the N terminus of histone H3 at lysine 4 (H3K4me and H3K4me2), changes associated with active promoters and either latent or active enhancers (Forneris et al., 2005; Shi et al., 2004; Whyte et al., 2012). Deletion of Lsd1 in mice causes embryonic lethality, thus suggesting a crucial role for Lsd1 in many developmental events Several lines of evidence suggest that Lsd1 regulates the maintenance of pluripotency and/or differentiation of multiple cell lineages (Chen et al., 2016; Laurent et al., 2015; Sun et al., 2010: Whyte et al., 2012). Based on this role we tested whether Lsd1 inhibitors might open and transcriptionally activate Wnt targets.

Herein, we show that targeting Lsd1 can promote derepression of genes required for hair cell differentiation in the neonatal and adult cochlea. We found that inhibition of Lsd1 in the new born cochlea enhanced responsiveness to Wnt signaling by increasing active marks toward the more permissive H3K4me2 at the Atoh1 locus and that Lsd1 inhibition and Wnt activation in the adult could also activate expression of Atoh1.

As shown herein, manipulation of the epigenetic modifier, Lsd1, can alter histone marks on a silenced gene leading to a specific change in cell phenotype and induction of progenitor cell differentiation to a hair cell. Lsd1 inhibition relieved Atoh1 gene repression, led to increased transcription of the bHLH transcription factor, and induced differentiation of the Lgr5-positive progenitor cells. Atoh1 is silenced soon after birth, but epigenetic changes induced by Lsd1 inhibition increased its expression, resulting in the differentiation of hair cells. Since we only saw this effect when LSD1 inhibition was combined with the stimulation of Wnt signaling, and we knew that Wnt signaling was key to development and regeneration of hair cells as well as expression of Atoh1, we hypothesize that Lsd1 normally keeps Wnt targets silenced/poised so that when Lsd1 is inhibited, the supporting cells become responsive to Wnt. Thus Lsd1 inhibition allows reversal of the Wnt blockade and the increased Atoh1 expression that results from the inhibition of LSD1 has an effect on the fate of progenitor cells in the cochlea—i.e. transcription factors downstream of Wnt are poised but become active. Lsd1 inhibition allows Atoh1 expression in both the new born and adult cochlea, where Atoh1 is silenced. We show in organoids prepared from newborn mice that this effect is mediated by enrichment of methylation of histones at the promoter and enhancer regions of the Atoh1 gene. Although we were unable to do this analysis in the adult as ChIP analysis requires more cells than can be obtained from the adult cochlea, we hypothesize that epigenetic modifications may have accounted for the increase in Atoh1 expression after Lsd1 inhibition in the adult and that the decline in adult regenerative capacity is due to the silencing of target genes required for hair cell differentiation.

Lsd1 was initially identified as a component of the HDAC-containing, Co-REST transcriptional repressor complex (Ballas et al., 2001). However, it was subsequently characterized as a histone demethylase, specific for removing methyl groups from the N-terminus of histone H3 at lysine 4 (H3K4me and H3K4me2), changes associated with active promoters and either latent or active enhancers (Forneris et al., 2005: Shi et al., 2004: Whyte et al., 2012). Conversely, when Lsd1 is bound to nuclear hormone receptors, it functions as a co-activator by mediating the demethylation of the repressive H3K9me2 mark (Metzger et al., 2005). Deletion of Lsd1 in mice causes embryonic lethality, thus suggesting a crucial role for Lsd1 in many developmental events. Notably, several lines of evidence suggest that Lsd1 regulates the maintenance of pluripotent cells and the differentiation of multiple cell lineages (Chen et al., 2016: Laurent et al., 2015: Sun et al., 2010; Whyte et al., 2012). Recruitment of Lsd1 to developmental genes has previously been reported during stem cell differentiation, but the underlying mechanism is elusive (Adamo et al., 2011: Chen et al., 2016). Specifically, some studies have implicated roles for Co-REST, a repressive complex and/or the presence of transcription factors at bivalent domains to influence Lsd1 recruitment to target genes (Adamo et al., 2011: Yamada et al., 2010). Interestingly, Gfi1 (also a hair cell-specific gene), is known to recruit Lsd1 to form a repressive complex. With Lsd1 depletion, these Gfi1 targets are derepressed and the levels of H3K4me2 are enhanced at target promoters (Kerenyi et al., 2013: Maiques-Diaz et al., 2018).

In the developing inner ear, the onset of Atoh1 expression occurs at E13.5 (Lumpkin et al., 2003). Its expression is accompanied by dynamic changes in bivalent (H3K4me3/H3K27me3), active (H3K9ac) and repressive (H3K9me3) histone marks at the Atoh1 locus, correlating with the onset of Atoh1 expression and moving towards repressive changes during its decline in the postnatal period (Stojanova et al., 2015). Previous ChIP-Seq analyses have revealed that Lsd1 occupies the promoter region of the Atoh1 gene in embryonic and neural stem cells, thus implicating Lsd1 in the regulation of Atoh1 transcription (Wang et al., 2016: Whyte et al., 2012). We first showed that Lsd1 interacted with the Atoh1 locus and that Lsd1 inhibiting drugs and Lsd1 deletion increased hair cells in organoids, specifically at the distal promoter and 3′ end of the enhancer region. To further examine the role of Lsd1 in Atoh1 regulation, we employed a nuclease-inactivated dCas9 fused to Lsd1 together with short guide RNA sequences to target the Atoh1 promoter or 3′ enhancer region of cochlear progenitor cells (Kearns et al., 2015). Significant Atoh1 downregulation upon targeting either the promoter or the 3′ end of the enhancer, and consequent repression of hair cell differentiation markers including Myo7a was observed, thus indicating the potential for Atoh1 to be activated using epigenetic modifiers. In the noise-exposed adult, we showed that treatment with pargyline and CHIR99021 increased the expression of Atoh1.

Since Lsd1 inhibition increased the expression of Wnt downstream genes in response to Wnt signaling we surmised that these genes were kept in a poised state and could be converted to a responsive state by Lsd1 inhibition such that they responded to the stimulation of Wnt signaling. As Atoh1 expression is modulated by increased β-catenin levels (Shi et al., 2010), we initially hypothesized that Lsd1 inhibition might be enhancing expression of Atoh1 through direct activation of the Wnt pathway. Several studies had implicated Lsd1 in regulating Wnt signaling suggesting that inhibiting Lsd1 could activate the pathway by upregulating components of the pathway or acting directly on levels of β-catenin (Chen et al., 2016; Lei et al., 2015: Zhou et al., 2016). While our RNA-sequencing data revealed upregulation of Wnt target genes with Lsd1 inhibition, no significant difference in the levels of active β-catenin or Tcf/Lef activity was observed in our study, thus indicating that the effect of Lsd1 on the Wnt pathway was not responsible for the increased hair cell differentiation observed. Our studies showed however that this was not a significant contributor to the effect of pargyline and rather the effect on Wnt downstream targets accounted for the upregulation relative to CHIR99021. Thus the effect of Lsd1 inhibition was not due to an additive effect to Wnt signaling but was an epigenetic effect on Wnt downstream targets. Atoh1 upregulation in the adult becomes significant with pargyline indicating that Lsd1 regulates Wnt downstream targets negatively and its inhibition allows for expression which in concert with the newborn progenitor cell results was likely due to enrichment of activating epigenetic marks on Wnt downstream target genes like the Atoh1 gene.

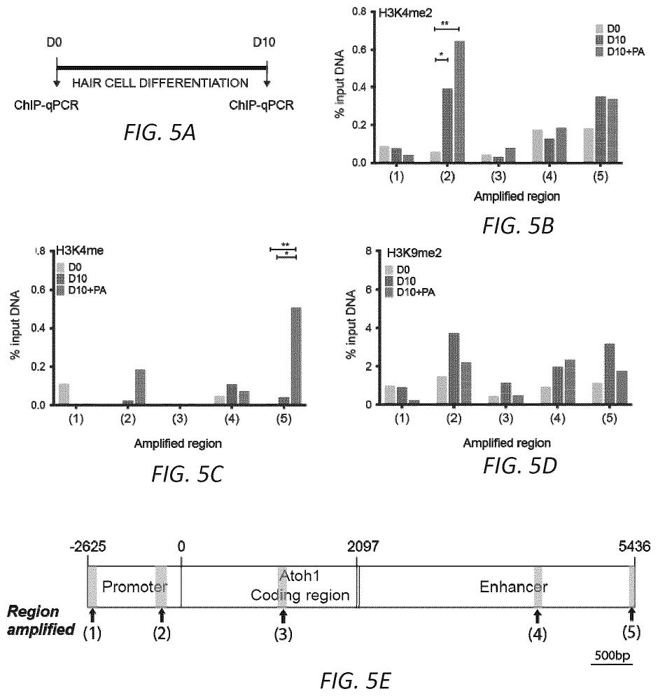

As Lsd1 is an important histone modifier, actively involved in maintaining the balance between H3K4 and H3K9 methylation at target genes (Adamo et al., 2011; Bernstein et al., 2006), we next examined the enrichment of these epigenetic marks on the Atoh1 locus. We found that the levels of H3K4me2 at the promoter and enhancer regions of the Atoh1 gene were significantly increased after treatment with pargyline. The levels of H3K9me2 on the Atoh1 gene were not significantly altered in the undifferentiated or differentiated cells (FIG. 5D). These data indicated that the effects of Lsd1 were limited to H3K4 marks in these cells and that the increased expression of Atoh1 after Lsd1 inhibition was due to their increased level in the presence of pargyline. These data also suggest that H3K9me2 demethylation which is carried out in the CNS by Lsd1 isoform 8a (Laurent et al., 2015: Metzger et al., 2005; Zibetti et al., 2010) was not a major contributor to gene silencing in the cochlea.

Our data reveal that Lsd1 depletion both genetically and pharmacologically can potentiate hair cell differentiation. The potential for epigenetic modifiers to reverse the chromatin assembly of bivalent genes from a heterochromatin to euchromatin state and activate transcription is well established. Inhibition of LSD1 had a more striking effect than any of the other treatments we found in our original work in organoids from the cochlea (McLean et al., 2017). We show here that the effects of Lsd1, valproic acid and pargyline were distinct, where valproic acid was the most effective on proliferation whereas pargyline created the most hair cells.

The role we find here for Lsd1 in the inner ear has been seen in recent findings in the chick although Lsd1 inhibition downregulated otic specific genes leading to a significant reduction in the size of the otic vesicle (Ahmed and Streit, 2018). The authors attributed this effect to the binding of Lsd1 to another transcription factor cMyb, with the loss of this interaction causing a reversal of the transcription activating effect of Lsd1 on otic genes. These discrepant findings may also be related to the fact that the specific Lsd1 isoforms present in mammals (including Lsd1+8a) are absent in other vertebrates including chick (Laurent et al., 2015: Zibetti et al., 2010), therefore the underlying mechanism of Lsd1 in these systems may differ. A role for Lsd1 in regulating inner ear neural differentiation (Patel et al., 2018) was however similar to the role in cellular differentiation found here. Lsd1 was observed to interact with Pax2 to form a repressive NuRD complex and suppress neural differentiation in an otic neural progenitor cell line. Pharmacological inhibition of Lsd1 reversed this repressive effect, mediated by an increase in H3K4me2 marks at promoters of sensory neural genes. Consistent with our findings, these studies have emphasized the importance of Lsd1 in inner ear development and sensory cell differentiation.

In neonates, a significant increase in supporting cell proliferation and hair cell differentiation was observed in the cochlea after treatment with pargyline and CHIR99021. In addition, acute treatment of noise-deafened mice resulted in a marginal improvement in ABR thresholds≤25-30 dB SPL at the low cochlear frequencies in some of the animals. Histological analyses of cochlear tissue of the treated mice revealed “new” hair cells derived from precursor supporting cells, marked by lineage tracing. Analysis of the transcriptional changes in the mice exposed to noise and treated with pargyline and CHIR99021 showed a significant increase in Atoh1 in the treated ears, thus revealing the potential for Lsd1 to activate Atoh1 expression in the deaf adult cochlea.

The hair cells and supporting cells in the mammalian cochlea are post-mitotic, and damage to these cells is thought to be permanent. By establishing a role for Lsd1 in regulating cochlear supporting cell proliferation and differentiation in the adult cochlea, this work enables replacement of damaged cells. As shown herein, manipulation of epigenetic marks, such as through Lsd1 or HDAC inhibition, in combination with an agonist of Wnt signaling, is a promising approach to facilitating regeneration in the adult ear.

Thus, described herein are methods for promoting regeneration of cochlear hair cells, by administering a combination of a Wnt agonist, e.g., a GSK3β inhibitor, and a KDM inhibitor.

### Methods of Treatment

In some embodiments, the present disclosure provides novel therapeutic strategies for treating hearing loss associated with a loss of vestibular hair cells (e.g., cochlear hair cells in the inner ear) or balance loss associated with a loss of vestibular hair cells, (i.e., conditions that would benefit from an increased proliferation and differentiation of inner ear supporting cells (e.g., Lgr5+ inner ear supporting cells)). In some embodiments, such strategies can promote an increase in the proliferation of inner ear supporting cells (e.g., Lgr5+ inner ear supporting cells) and/or an increase in the differentiation of the inner ear supporting cells (e.g., Lgr5+ inner ear supporting cells) into inner ear hair cells (e.g., Atoh1+ inner ear hair cells), thereby promoting the expansion and differentiation of a target cell into a mature cell of the inner ear, e.g., an auditory hair cell. In some embodiments, the methods and compositions described herein promote differentiation of target cells (e.g., inner ear supporting cells (e.g., Lgr5+ inner ear supporting cells)) to or towards mature cells of the inner ear, e.g., auditory hair cells (e.g., inner ear hair cells (e.g., Atoh1+ inner ear hair cells)) without promoting substantial cellular proliferation. In some embodiments, the methods and compositions described herein promote proliferation of target cells (e.g., inner ear supporting cells (e.g., Lgr5+ inner ear supporting cells)) without promoting substantial cellular proliferation.

In some embodiments, the present invention can be used to treat hair cell loss and any disorder that arises as a consequence of cell loss in the ear, such as hearing impairments, deafness, and vestibular disorders, for example, by promoting differentiation (e.g., complete or partial differentiation) of one or more cells (e.g., inner ear supporting cells (e.g., Lgr5+ inner ear supporting cells)) into one or more cells capable of functioning as sensory cells of the ear, e.g., hair cells (e.g., inner ear hair cells (e.g., Atoh1+ inner ear hair cells)).

In some embodiments, the hearing loss is sensorineural hearing loss, which can result from damage or malfunction of the cochlea, e.g., loss of or damage to the sensory epithelium resulting in loss of hair cells.

In some embodiments, the hearing loss can be for any reason, or as a result of any type of event. For example, because of a genetic or congenital defect: for example, a human subject can have been deaf since birth, or can be deaf or hard-of-hearing as a result of a gradual loss of hearing due to a genetic or congenital defect. In another example, the hearing loss can be a result of a traumatic event, such as a physical trauma to a structure of the ear, or a sudden loud noise, or a prolonged exposure to loud noises. For example, prolonged exposures to concert venues, airport runways, and construction areas can cause inner ear damage and subsequent hearing loss.

In some embodiments, hearing loss can be due to chemical-induced ototoxicity, wherein ototoxins include therapeutic drugs including antineoplastic agents, salicylates, quinines, and aminoglycoside antibiotics, contaminants in foods or medicinals, and environmental or industrial pollutants. In some embodiments, hearing loss can result from aging.

Described herein are methods of treating a subject having hearing loss or balance loss, in which a therapeutically effective amount of: (i) a KDM inhibitor and (ii) a Wnt signaling activator, e.g., as set forth in Table A, are administered to the subject, e.g., to the ear of a subject. In some embodiments, the subject is at least 1 month old, e.g., at least 2 months, 3 months, 6 months, 12 months, 18 months, 2 years, 5 years, 6 years, 10 years, 16 years, 30 years, 40 years, 45 years, 50 years, 55 years, 60 years, 65 years, or 70 years old. In general, the subject is a mammal, e.g., a human or a veterinary subject (e.g., a dog, cat, horse, or other farm, zoo, or household animal).

In some embodiments, the methods promote proliferation and/or differentiation of inner ear supporting cells (e.g., Lgr5+ inner ear supporting cells) into inner ear hair cells (e.g., Atoh1+ inner ear hair cells).

Also provided are methods of treating a subject having hearing loss or balance loss, in which a therapeutically effective amount of: (i) a KDM inhibitor and (ii) a Wnt signaling activator, e.g., as set forth in Table A, are administered to the subject, e.g., to the ear of a subject.

In some embodiments of the methods of treating a subject having hearing loss or balance loss, the KDM inhibitor and Wnt signaling activator are administered systemically or to the ear of the subject, e.g., transtympanically to the middle ear of the subject. In some embodiments, the KDM inhibitor and Wnt signaling activator are administered together or separately, either immediately after, or within weeks, months or years of the onset of the hearing loss or balance disorder.

In some embodiments of the methods of treating a subject described herein, the subject is a human.

In general, compounds and methods described herein can be used to generate hair cell growth (e.g., Atoh1+ inner ear hair cell growth) in the ear and/or to increase the number of hair cells in the ear (e.g., in the inner, middle, and/or outer ear). For example, the number of hair cells in the ear can be increased about 2-, 3-, 4-, 6-, 8-, or 10-fold, or more, as compared to the number of hair cells before treatment. This new hair cell growth can effectively restore or establish at least a partial improvement in the subject's ability to hear. For example, administration of an agent can improve hearing loss by about 5, 10, 15, 20, 40, 60, 80, 100% or more.

Where appropriate, following treatment, a human can be tested for an improvement in hearing or in other symptoms related to inner ear disorders. Methods for measuring hearing are well-known and include pure tone audiometry, air conduction, and bone conduction tests. These exams measure the limits of loudness (intensity) and pitch (frequency) that a human can hear. Hearing tests in humans include behavioral observation audiometry (for infants to seven months), visual reinforcement orientation audiometry (for children 7 months to 3 years) and play audiometry for children older than 3 years. Oto-acoustic emission testing can be used to test the functioning of the cochlea hair cells, and electro-cochleography provides information about the functioning of the cochlea and the first part of the nerve pathway to the brain. In some embodiments, treatment can be continued with or without modification or can be stopped.

TABLE A
Wnt Agonists

| Compound | Target |
|---|---|
| CHIR-99021 | GSK-3β |
| CHIR-98023 | GSK-3β |
| CHIR-99030 | GSK-3β |
| Hymenialdisine | GSK-3β |
| debromohymeialdisine | GSK-3β |
| dibromocantherelline | GSK-3β |
| Meridianine A | GSK-3β |
| alsterpaullone | GSK-3β |
| cazapaullone | GSK-3β |
| Aloisine A | GSK-3β |
| NSC 693868 | GSK-3β |
| (1H-Pyrazolo[3,4-b]quinoxalin-3- | |
| amine) | |
| Indirubin-3′-oxime | GSK-3β |
| (Indirubin-3′-monoxime; 3-[1,3- | |
| Dihydro-3-(hydroxyimino)-2H-indol-2- | |
| ylidene]-1,3-dihydro-2H-indol-2-one) | |
| A 1070722 | GSK-3β |
| (1-(7-Methoxyquinolin-4-yl)-3-[6- | |
| (trifluoromethyl)pyridin-2-yllurea) | |
| L803 | GSK-3β |
| L803-mts | GSK-3β |
| TDZD8 | GSK-3β |
| NP00111 | GSK-3β |
| HMK-32 | GSK-3β |
| Manzamine A | GSK-3β |
| Palinurin | GSK-3β |
| Tricantin | GSK-3β |
| IM-12 | GSK-3β |
| (3-(4-Fluorophenylethylamino)-1- | |
| methyl-4-(2-methyl-1H-indol-3-yl)-1H- | |
| pyrrole-2,5-dione) | |
| NP031112 | GSK-3β |
| NP00111 | GSK-3β |
| NP031115 | GSK-3β |
| VP 2.51 | GSK-3β |
| VP2.54 | GSK-3β |
| VP 3.16 | GSK-3β |
| VP 3.35 | GSK-3β |
| HLY78 | Axin |
| (4-Ethyl-5,6-Dihydro-5-methyl- | |
| [1,3]dioxolo[4,5-j]phenanthridine, 4-Ethyl-5- | |
| methyl-5,6-dihydro-[1,3]dioxolo[4,5- | |
| j]phenanthridine) | |
| WAY-262611 | Dickkopf-1 (DKK1) |
| ((1-(4-(Naphthalen-2-yl)pyrimidin-2- | |
| yl)piperidin-4-yl)methanamine)) | |
| BHQ880 | DKK1 |
| NCI8642 | DKK1 |
| gallocyanine dyes | DKK1 |
| Compounds 3-8 | secreted frizzled-related |
| (Moore et al., J. Med. Chem., 2009; | protein 1 (sFRP-1) |
| 52: 105) | |
| WAY-316606 | sFRP-1 |

### KDM Inhibitor

In some embodiments, the KDM inhibitor is selected from the group consisting of tranylcypromine ((trans-2-phenylcyclopropyl-1-amine, trans-2-PCPA)) and analogs thereof (e.g., with one or more substitutions, e.g., at the benzene ring or at the amine position (e.g., ORY-1001 (rel-N1-[(1R,2S)-2-phenylcyclopropyl]-1,4-cyclohexanediamine, dihydrochloride): S2101 ((1R,2S)-rel-2-[3,5-Difluoro-2-(phenylmethoxy)phenyl]cycloprpanamine hydrochloride); or GSK-LSD1 (rel-N-[(1R,2S)-2-Phenylcyclopropyl]-4-Piperidinamine hydrochloride)); 2,4-pyridinedicarboxylic acid (2,4-PDCA); Pargyline (N-Methyl-N-propargylbenzylamine) or Pargyline hydrochloride (N-Methyl-N-propargylbenzylamine hydrochloride); or C12 ((E)-N′-(1-(5-chloro-2-hydroxyphenyl)ethylidene)-3-(morpholinosulfonyl)benzohydrazide): 2,4-pyridinedicarboxylic acid (2,4-PDCA): 5-Carboxy-8-hydroxyquinoline (IOX1) and n-octyl ester thereof. In some embodiments, the KDM inhibitor is selected from the group consisting of tranylcypromine (trans-2-phenylcyclopropyl-1-amine, trans-2-PCPA, TCP) and analogs thereof (e.g., with one or more substitutions, e.g., at the benzene ring or at the amine position (e.g., ORY-1001 (rel-N1-[(1R,2S)-2-phenylcyclopropyl]-1,4-cyclohexanediamine, dihydrochloride): S2101 ((1R,2S)-rel-2-[3,5-Difluoro-2-(phenylmethoxy)phenyl]cycloprpanamine hydrochloride); or GSK-LSD1(rel-N-[(1R,2S)-2-Phenylcyclopropyl]-4-Piperidinamine hydrochloride))): 2,4-pyridinedicarboxylic acid (2,4-PDCA): 5-Carboxy-8-hydroxyquinoline (IOX1) n-octyl ester thereof, and Pargyline (N-Methyl-N-propargylbenzylamine) or pargyline hrdyochloride (N-Methyl-N-propargylbenzylamine hydrochloride). In some embodiments, the KDM inhibitor is C12 (HCI-2509, or (E)-N′-(1-(5-chloro-2-hydroxyphenyl)ethylidene)-3 (morpholinosulfonyl)benzohydrazide).

The chemical structures of C12, ORY-1001, PA hydrochloride, S2101, TCP and GSK-LSD1 are provided below:

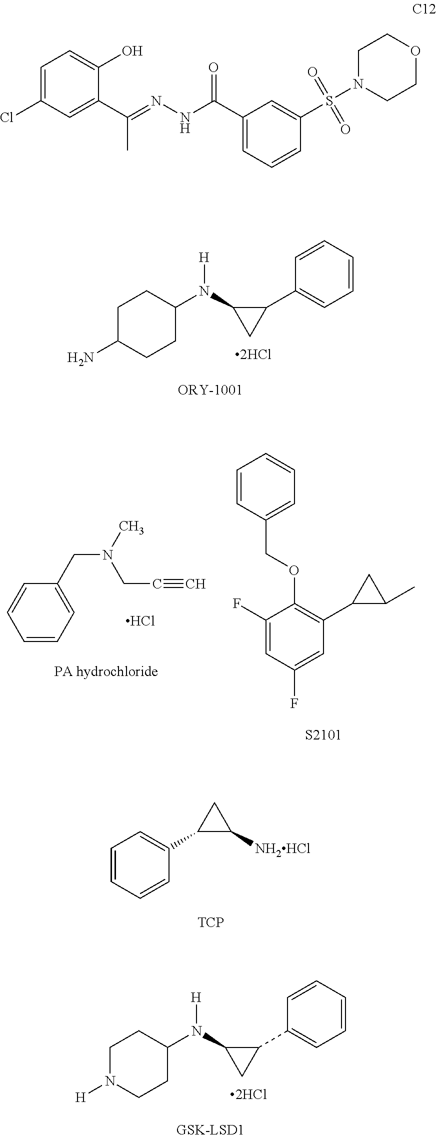

### Derivatives

In some embodiments, derivatives of the compounds (e.g., the KDM inhibitors or Wnt agonists listed herein or known in the art) described herein can also be used. A derivative of a compound is a small molecule that differs in structure from the parent compound, but retains the ability to promote the proliferation and expansion of inner ear supporting cells (e.g., Lgr5+ inner ear supporting cells) or to promote the differentiation of inner ear supporting cells (e.g., Lgr5+ inner ear supporting cells) into inner ear hair cells (e.g., Atoh1+ inner ear hair cells). A derivative of a compound may change its interaction with certain other molecules or proteins relative to the parent compound. A derivative of a compound may also include a salt, an adduct, or other variant of the parent compound. In some embodiments of the invention, any derivative of a compound described herein (e.g., any of the KDM inhibitors or Wnt agonists listed herein or known in the art) may be used instead of the parent compound in a method or composition described herein. In some embodiments, any derivative of a KDM inhibitor or Wnt agonist listed herein or known in the art may be used in a method of treating a subject, or of producing an expanded population of inner ear supporting cells, or of promoting differentiation of a population of inner ear supporting cells into a population of inner ear hair cells.

### Pharmaceutical Compositions

In some embodiments, one or more compounds as described herein can be formulated as one or more pharmaceutical compositions. Pharmaceutical compositions containing one or more compounds as described herein can be formulated according to the intended method of administration.

One or more compounds as described herein can be formulated as pharmaceutical compositions for direct administration to a subject. Pharmaceutical compositions containing one or more compounds can be formulated in a conventional manner using one or more physiologically acceptable carriers or excipients. For example, a pharmaceutical composition can be formulated for local or systemic administration, e.g., administration by drops (e.g., otic drops) or injection into the ear, insufflation (such as into the ear), intravenous, topical, or oral administration.

The nature of the pharmaceutical compositions for administration is dependent on the mode of administration and can readily be determined by one of ordinary skill in the art. In some embodiments, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral.

A pharmaceutical composition can be formulated in various ways, according to the corresponding route of administration. For example, liquid solutions can be made for administration by drops into the ear, for injection, or for ingestion: gels or powders can be made for ingestion or topical application. Methods for making such formulations are well known and can be found in, for example, Remington: The Science and Practice of Pharmacy, 22^{nd} Ed., Allen, ed., Mack Publishing Co., Easton, Pa., 2012.

One or more of the compounds can be administered, e.g., as a pharmaceutical composition, directly and/or locally by injection or through surgical placement, e.g., to the inner ear. The amount of the pharmaceutical composition may be described as the effective amount or the amount of a cell-based composition may be described as a therapeutically effective amount. Where application over a period of time is advisable or desirable, the compositions of the invention can be placed in sustained released formulations or implantable devices (e.g., a pump).

Alternatively or in addition, the pharmaceutical compositions can be formulated for systemic parenteral administration by injection, for example, by bolus injection or continuous infusion. Such formulations can be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use.

In addition to the formulations described previously, the compositions can also be formulated as a depot preparation. Such long acting formulations can be administered by implantation (e.g., subcutaneously). Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

Pharmaceutical compositions formulated for systemic oral administration can take the form of tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate): lubricants (for example, magnesium stearate, talc or silica): disintegrants (for example, potato starch or sodium starch glycolate): or wetting agents (for example, sodium lauryl sulphate). The tablets can be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats): emulsifying agents (for example, lecithin or acacia): non-aqueous vehicles (for example, almond oil, oily esters, ethyl alcohol or fractionated vegetable oils): and preservatives (for example, methyl or propyl-p-hydroxy benzoates or sorbic acid). The preparations may also contain buffer salts, flavoring, coloring and sweetening agents as appropriate. Preparations for oral administration may be suitably formulated to give controlled release of the active compound.

In some embodiments, the therapeutic compounds are prepared with carriers that will protect the therapeutic compounds against rapid elimination from the body, such as a controlled release formulation, including implants and microencapsulated delivery systems. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811. Nanoparticles, e.g., poly lactic/glycolic acid (PLGA) nanoparticles (see Tamura et al., Laryngoscope. 2005 November; 115(11):2000-5: Ge et al., Otolaryngol Head Neck Surg. 2007 October; 137(4):619-23: Horie et al., Laryngoscope. 2010 February; 120(2):377-83: Sakamoto et al., Acta Otolaryngol Suppl. 2010 November; (563):101-4) can also be used.

Such polymers and hydrogels are known in the art, see, e.g., Paulson et al., Laryngoscope. 2008 April; 118(4):706-11 (describing a chitosan-glycerophosphate (CGP)-hydrogel based drug delivery system): other carriers can include thermo-reversible triblock copolymer poloxamer 407 (see, e.g., Wang et al., Audiol Neurootol. 2009; 14(6): 393-401. Epub 2009 Nov. 16, and Wang et al., Laryngoscope. 2011 February; 121(2):385-91): poloxamer-based hydrogels such as the one used in OTO-104 (see, e.g., GB2459910; Wang et al., Audiol Neurotol 2009; 14:393-401; and Piu et al., Otol Neurotol. 2011 January; 32(1): 171-9); Pluronic F-127 (see, e.g., Escobar-Chavez et al., J Pharm Pharm Sci. 2006; 9(3):339-5); Pluronic F68, F88, or F108; polyoxyethylene-polyoxypropylene triblock copolymer (e.g., a polymer composed of polyoxypropylene and polyoxyethylene, of general formula E106 P70 E106: see GB2459910, US20110319377 and US20100273864): MPEG-PCL diblock copolymers (Hyun et al., Biomacromolecules. 2007 April; 8(4):1093-100. Epub 2007 Feb. 28): hyaluronic acid hydrogels (Borden et al., Audiol Neurootol. 2011:16(1): 1-11): gelfoam cubes (see, e.g., Havenith et al., Hearing Research, February 2011; 272(1-2):168-177); and gelatin hydrogels (see, e.g., Inaoka et al., Acta Otolaryngol. 2009 April; 129(4):453-7); other biodegradable, biocompatible polymers can be used, such as ethylene vinyl acetate, polyanhydrides, polyglycolic acid, collagen, polyorthoesters, and polylactic acid. Tunable self-assembling hydrogels made from natural amino acids L and D can also be used, e.g., as described in Hauser et al e.g. Ac-LD6-COOH (L) e.g. Biotechnol Adv. 2012 May-June; 30(3):593-603. Such formulations can be prepared using standard techniques, or obtained commercially, e.g., from Alza Corporation and Nova Pharmaceuticals, Inc.

In some embodiments, the pharmaceutical compositions described herein can include one or more of the compounds formulated according to any of the methods described above, and one or more cells obtained to the methods described herein.

### EXAMPLES

The invention is further described in the following examples, which do not limit the scope of the invention described in the claims.

### Methods

The following methods and materials were used in the Examples below.

Details of antibodies. The following table provides details of antibodies used in the Examples.

| Antibody | Company | Cat # |
|---|---|---|
| H3K4me | Diagenode | C15410194 |
| H3K4me2 | Active motif | 39679 |
| H3K9me2 | Active motif | 39375 |
| Lsd1 | Abcam | Ab17721 |
| Myo7a | Proteus Biosciences | 25-6790 |
| Phalloidin | Thermofisher | A22284 |
| Sox2 | R&D | AF2018 |

Mouse Strains. To assess the effects of the Lsd1 inhibitors in LCPs, Lgr5 cells, the Lgr5-EGFP-IRES-Cre-ER mice [The Jackson Laboratory, strain 8875; (Barker et al., 2007)] and Atoh1-nGFP mice [provided by Dr. Jane Johnson (Lumpkin et al., 2003)] were used.

The transgenic Lsd1^{fl/fl} mice (The Jackson Laboratory, strain 023969; (Kerenyi et al., 2013)) crossed to Sox2-CreER (The Jackson Laboratory, strain 017593) and Atoh1-nGFP were also used to examine the effect of Lsd1 on hair cell differentiation in vitro. The analysis was performed using only heterozygous Lsd1^{+/-} mice due to the homozygous animals generating reduced numbers of pups insufficient to generate organoids.

For the in vivo experiments, transgenic mice with a Sox2-CreER reporter strain (Mtmg, The Jackson Laboratory, strain 007576) were used to perform lineage tracing of Sox2-positive cells since (Fujioka et al., 2015). All animal studies were conducted under an approved institutional protocol according to National Institutes of Health guidelines.

Isolation and expansion of inner ear Lgr5+ cells. A recently published protocol was used to derive organoids from inner ear stem cells [also referred to as Lgr5+ cochlear progenitors (LCPs) (McLean et al., 2017). Briefly, the organ of Corti was dissected from neonatal mice (postnatal days 2-5) in Hank's balanced salt solution and treated with Cell Recovery Solution (Corning) for 1 hour. The cochlear epithelium was then peeled from the underlying mesenchyme, collected and treated with TrypleE for 20 minutes at 37° C. The single cells were mechanically triturated, filtered (40 μM cell strainer) and suspended in a Matrigel (Corning) dome for 3D culture. The cells were seeded in a 24-well plate, specifically at one cochlea per well of a 24-well plate. The cells were bathed in a serum free expansion media containing 1:1 mixture of DMEM and F12, supplemented with Glutamax (GIBCO), N2, B27 (ThermoFisher Scientific), EGF (50 ng/ml: Chemicon), bFGF (50 ng/ml; Chemicon), IGF-1 (50 ng/ml: Chemicon), and small molecules CHIR99021 (3 μM), valproic acid (1 mM) and phosphor-vitamin C (100 μg/mL).

Differentiation of LCPs into a hair cell lineage. After 10 days, the expansion media was removed and replaced with a serum-free 1:1 mixture of DMEM and F12, supplemented with Glutamax (GIBCO), N2, and B27, (ThermoFisher Scientific) and various combinations of drugs (LY411575, 10 μM, CHIR99021, 3 μM: pargyline, 10 μM) for an additional 10 days. To analyze the effect of the Lsd1 inhibitors, the LCPs were treated with the small molecules combined with a differentiation drug cocktail, pargyline and CHIR99021 or CHIR99021 at 1, 2, 4, and 10 days (McLean et al., 2017).

For the drug screening experiments, the expanded cells were passaged into a 96 well plate to test varying drug combinations. All experiments were done in triplicate at minimum. Atoh1-nGFP cells were quantified after 10 days in culture. The cell colonies were incubated in Cell Recovery Solution for 1 hour and dissociated into single cells using TrypleE. The total cell number and percentage of GFP+ cells were quantified using fluorescence activated cell sorting (FACS).

TABLE 1
Details of drugs used

| LSD1 inhibitor drugs | Company | Cat # |
|---|---|---|
| C12 (HCl-2509) | Xcessbio | M60160 |
| CHIR99021 | Cayman | 13122 |
| LY411575 | Cayman | 16162 |
| ORY-1001 | Cayman | 19136 |
| PA hydrochloride | Sigma-Aldrich | P8013 |
| S2101 | Millipore Sigma | 489477 |
| TCP | Tocris | 3852 |

RNA extraction and PCR. For the LCPs, the organoids were treated with Cell Recovery Solution for at 1 hour and TrypleE for 20 minutes, then frozen in RLT buffer. RNA extractions were performed using the RNeasy Micro Kit (QIAGEN) and cDNA generated using ImProm-II Reverse Transcription Kit (Promega). TaqMan Real-Time PCR was performed in triplicates using the TaqMan Gene Expression Master Mix (ThermoFisher Scientific) on a StepOne Real-Time PCR machine (ThermoFisher Scientific). Ordinary One-way Anova was used to assess statistical significance. A minimum of three biologically distinct samples were analyzed for each condition.

RNA sequencing. After RNA extractions were performed using the RNeasy Micro Kit (QIAGEN), the library preparation and RNA-sequencing was performed at the Dana Farber Cancer Institute Molecular Biology Core Facility. In brief, cDNA was synthesized from 2.5 ng of RNA using SMARTer V4 Kit (Clontech). Following fragmentation using M220 Focused-Ultrasonicator (Covaris), 2 ng of sheared cDNA was taken for library preparation using ThruPLEX DNA-seq kit (Rubicon Genomics). NextSeq500 Single-End 75 bp (SE75) Sequencing (Illumina) was performed on all 12 samples in one lane after they were indexed and pooled in equimolar to ensure 20-30 million reads per sample. Reads were aligned to mm10 augmented with Ensembl gene build 75 with STAR v2.5.2b (Dobin et al., 2013). Reads and alignments were assessed for quality using FastQC. Differential expression was evaluated using DESeq2. Genes were called differentially expressed when the absolute value of the fold change was >1.25 and FDR, was smaller than 0.05. Subsequent pathway analysis was performed using Ingenuity Pathway Analysis software (Qiagen).

Chromatin immunoprecipitation. The colonies were treated with Cell Recovery solution for up to 2 hours and TrypleE for 30 minutes. The cells were fixed with 1% formaldehyde for 30 minutes at room temperature before termination with 0.1M Glycine. The cells were treated with lysis buffer (0.5M EDTA and 0.05% Triton-X 100) for 30 minutes on ice and then with nuclear extraction buffer (0.5M EDTA, 20% SDS, 1M Tris-HCl, pH 8) for 10 minutes. The cross-linked chromatin was sonicated for 30 minutes (30×30 seconds with 30 second intervals) and shearing quality confirmed by running 10 ul of the sample on a 1% agarose gel. The input sample used was 5% of the total chromatin. Lo-bind tubes containing Dyna-A and Dyna-B beads in PBS-BSA were incubated with 3 μg of Primary antibody for 6 hours-overnight with rotation at 4° C. The antibodies used were as follows H3K4me (ab8895: Abcam), H3K4me2 (ab77661: Abcam), H3K9me2 (ab1220; Abcam) and Lsd1 (ab17721: Abcam). The chromatin samples were treated with dilution buffer (1% Triton-X 100, 0.5M EDTA, 5M NaCl, 1M Tris-HCl, pH 8 and 1% Protease inhibitors×100) and incubated with the prepared beads for 6 hours-overnight. The beads were captured on a magnetic rack, washed with ChIP RIPA buffer (1M Hepes, 0.5M EDTA, 10% Na-deoxycholate, NP-40 and 5M LiCl: 6×10 minute washes), TE buffer (1M Tris-HCl and 0.5M EDTA: 2×10 minute washes), resuspended in reverse crosslinking solution (0.5M NaHCO₃ and 20% SDS) and incubated overnight at 65° C. DNA was recovered using the Active motif DNA extraction kit (58002: Active Motif). RT-qPCR analyses were performed on immunoprecipitated DNA using specific primers described in Table 2. The results were calculated and presented as relative fold enrichment over the input.

TABLE 2
Primers used in ChIP-qPCR

| Primers | | SEQ | | SEQ |
|---|---|---|---|---|
| for | | ID | | ID |
| ChIP-qPCR | Forward | NO: | Reverse | NO: |
| Distal | ACAGAGCGGG | 1 | CCTCGGGAGG | 2 |
| promoter | ACAGGTGGGT | | CCCCGGTTTA | |
| Proximal | CCCTCACTC | 3 | CGTGCGAGGA | 4 |
| promoter | AGGTCGCCT | | GCCAATCA | |
| Coding | ACATCTCCCA | 5 | GGGCATTTGG | 6 |
| region | GATCCCACAG | | TTGTCTCAGT | |
| Enhancer | ACACCGCTGT | 7 | CCTTCAGCTC | 8 |
| 1 | TGTTTTCCAG | | TCCCGGAAAT | |
| | AGT | | CAAA | |
| Enhancer | AGAGCGGCTG | 9 | GTGCGCTCAC | 10 |
| 2 | ACAATAGAGG | | TCAGCGAC | |

Western blot analysis. For protein collection, cells were washed twice with PBS and then lysed by lysis buffer (20 mM HEPES, 400 mM NaCl, 1 mM EDTA, 0.1% NP-40 and 10% Glycerol) supplemented with HALT protease and phosphatase inhibitors (Thermo Fisher Scientific, Waltham, MA). Lysates were then centrifuged at 13,000 g for 15 min at 4° C., and each supernatant was collected. Protein concentration was determined using the Pierce BCA Protein Assay Kit (Pierce Biotechnology, Rockford, IL). Proteins within each lysate were separated by SDS-PAGE (Bio-Rad Laboratories, Inc., Hercules, CA) and transferred to polyvinylidene difluoride membranes. After incubation with primary antibody, membranes were washed with Tris-buffered saline with Tween 20 and exposed to an appropriate horseradish peroxidase-conjugated secondary antibody (Santa Cruz Biotechnology, Santa Cruz, CA); immunoblot signals were detected with Pierce ECL Western blotting substrate or ECL plus (Thermo Fisher Scientific, Waltham, MA).

Luciferase assay. Wnt activity was assessed in inner ear organoids using lentiviral vector expressing Firefly luciferase under the control of 7 Tcf binding sites (Fuerer and Nusse, 2010). Cochlear progenitor cells were dissected from sensory epithelia of wild-type neonatal mice using the established protocol described above (McLean et al., 2017). Dissociated cells were resuspended in proliferation media contain poly brene (Sigma) and lentivirus reporter (7TFC, Addgene #24307) and plated in 96 well plate coated with matrigel for 12-16 hours, [adapted from (Maru et al., 2016)]. The following day, media (containing dead cells and virus) was aspirated and a top layer of matrigel applied to the adherent cells and allowed to solidify, followed by addition of proliferation media. Media was changed every 2 days for 10 days as organoids developed. At day 10, media was changed to differentiation conditions with removal of growth factors and treatment with drug combinations described above. Efficient infectivity of organoids was assessed with Zeiss Brightfield live microscope for mcherry signal which demonstrated high efficient infection of organoids. Luciferase was measured at 48 hours and normalized to total protein concentration.

Cochlear explant studies. Cochleae were dissected from postnatal day 2 Lgr5-GFP or Atoh1-nGFP mice and transferred to HBSS. The organ of Corti was isolated from the otic capsule and the basal hook portion was removed for optimal plating. The organ of Corti were plated on to a matrigel-coated (1:10 mixture of serum-free DMEM and matrigel) glass coverslips and cultured in a serum-free 1:1 mixture of DMEM and F12, supplemented with Glutamax, N2, and B27. For the treated explants, small molecule drugs were added to the media, whilst for the control cochlea, DMSO was added at the same concentrations used in the treatments (0.1-2.3%).

To assess supporting cell proliferation in culture, 10 μM of Edu was also added to the media. The organs were cultured in this media for three days and stained for Myo7a, Sox2 and Lgr5-GFP. The number of cells expressing Myo7a were counted within a 200 μM segment of the apical-mid region of the cochlea.

Acoustic exposure. Four-week-old mice were exposed to free field, awake and unrestrained, in a small reverberant chamber. Acoustic trauma was produced by a 2-hour exposure to 1-octave band of noise (8-16 kHz) presented at 118 dB SPL. The exposure stimulus was generated by a custom white noise source, filtered (Brickwall Filter with a 60 db/octave slope), amplified (Crown power amplifier), and delivered (JBL compression driver) through an exponential horn fitted securely to a hole in the top of a reverberant box. Sound exposure levels were measured at four positions within each cage using a 0.25 inch Brüel & Kjær condenser microphone: sound pressure was found to vary by <0.5 dB across these measurement positions.

ABR measurements. Auditory brain stem responses were measured in each animal at seven log-spaced frequencies (half-octave steps from 5.6 to 45.2 kHz) before and 1 day after noise exposure, 1 week and 1 month after surgery. Mice were anesthetized with ketamine (100 mg/kg, i.p.) and xylazine (20 mg/kg, i.p.). Needle electrodes were inserted at the vertex, pinna and tail (grounding electrode). ABRs were evoked with 5 ms tone pips. The response was amplified, filtered, and averaged in a Lab-VIEW-driven data acquisition system. Sound level was raised in 5 dB steps from ≥10 dB below threshold to <80 dB SPL. At each sound level, 1,024 responses were averaged (with alternated stimulus polarity), using an “artifact reject,” whereby response waveforms were discarded when peak-to-peak response amplitude exceeded 15 mV. ABR thresholds were defined as the lowest SPL level at which any wave could be detected, usually corresponding to the level step just below that at which the peak-to-peak response amplitude rose significantly above the noise floor (approximately 0.25 mV). When no response was observed at the highest sound level available, the threshold was designated as being 5 dB greater than that level so that statistical tests could be done.

Round window drug administration. We used four-week-old mice weighing 12-15 g. The animals were anesthetized with ketamine (20 mg/kg, intraperitoneally [i.p.]) and xylazine (100 mg/kg, i.p.) prior to surgery. An incision was made posterior to the pinna near the external meatus to expose the otic bulla and subsequently the round window niche. pargyline and CHIR99021 dissolved in DMSO were diluted in polyethylene glycol 400 (Sigma) to obtain final concentrations of 0.1 and 5 mM. This solution (total volume 1 ul) was injected into the round window niche of the left ear. Polyethylene glycol 400 with 10% DMSO was injected into the right ear as a control. Gelfoam was placed on the niche to maintain the solution, and the wound was closed.

Immunochemistry. The colonies or neonatal cochlear explants were fixed in 4% paraformaldehyde for 10-15 minutes at room temperature and then washed 3 times in PBS. For collection of mature cochleae, adult animals were sacrificed via CO₂ administration. The temporal bones were isolated, and a small opening was made at the apex of the cochlea. The temporal bones were immersed in 4% paraformaldehyde at 4° C. overnight with rotation and then decalcified in 0.1M EDTA (pH 7.4) for up to 3 days at room temperature. The cultures were blocked in 0.1% Triton X-100 and 10% heat inactivated donkey serum in PBS for 1 hour. The primary antibodies were diluted in blocking solution and applied overnight at 4° C. The primary antibodies and dilutions used are listed Table S1. The relevant Secondary antibodies (Alexa 488, Alexa Fluor 568, and Alexa Fluor 647 conjugated: Thermo Fisher Scientific) were diluted at 1:500 in PBS. Nuclei were visualized using DAPI (Vector Laboratories). Edu was labelled using the Click-iT EdU imaging kit (Thermo Fisher Scientific). The cochlear explants were treated with Edu (10 μM) for 3 days in culture and subsequently washed and stained following the kit guidelines. All staining was visualized and imaged using confocal microscopy (TCS, Leica).

### Example 1. Pharmacological and Genetic Inhibition of Lsd1 Potentiates Neonatal Hair Cell Differentiation

Stimulation of the Wnt pathway can activate supporting cell proliferation and hair cell differentiation in the neonatal organ of Corti, but this response is lost in adults (Chai et al., 2012: Shi et al., 2013; Shi et al., 2012). A possible explanation for this discrepancy is that Wnt target genes in adults are non-permissive or in a heterochromatin state, thus restricting hair cell differentiation. If that were the case, reversing this status could be necessary in order to stimulate the expression of Wnt target genes in adults. Here, we set out to investigate the effect of Lsd1, a well-established transcriptional repressor, associated with the Co-REST complex.

We generated inner ear organoids from Lgr5-expressing supporting cells of the cochlea utilizing our previously established procedure (McLean et al., 2017). The organoids comprise Lgr5-expressing cochlear progenitors cells (LCPs) capable of differentiating in high yield to hair cells and allow screening for genes or drugs that expand the LCPs or differentiate them to hair cells.

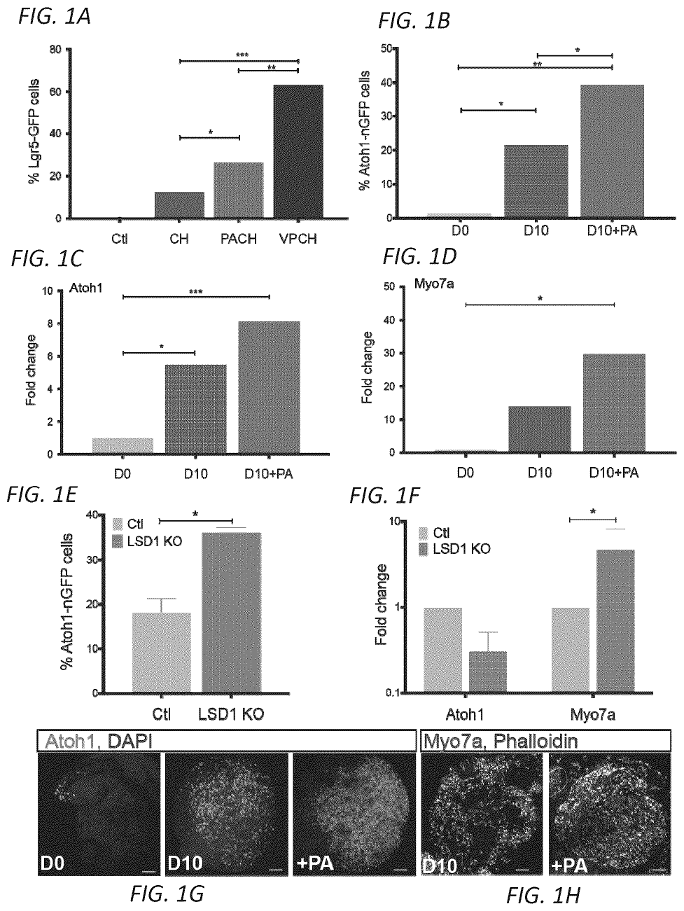

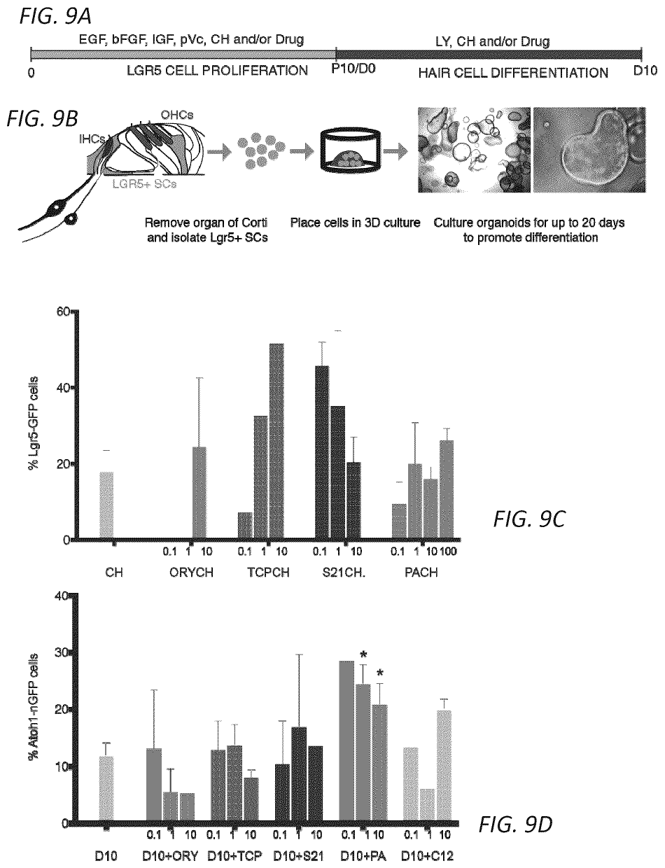

We screened a collection of Lsd1 inhibitors over the first 10 days in culture (FIGS. 9A and 9B) to examine their effect on proliferation of LCPs (FIGS. 9C and 9D). We previously reported that inhibition of HDAC, another component of the Co-REST complex, using valproic acid, increased LCP proliferation in vitro, above what could be achieved by activating the Wnt pathway only (McLean et al., 2017). Among the Lsd1 inhibitors tested, pargyline showed the most potent effect on promoting proliferation of Lgr5 cells: however it's effect was dependent on the presence of CHIR99021 (p<0.05*). In addition, the effect was significantly lower than that of the HDAC inhibitor, valproic acid (26 versus 63% p<0.01**: FIG. 1A) in increasing the cell division of the LCPs.

We next tested the effect of Lsd1 inhibition on hair cell differentiation. In this assay, after the cells were expanded into organoids during the initial proliferation phase, differentiation was initiated using the combination of CHIR99021 and LY411575 and allowed to progress for 10 days (DO to D10). Organoids were monitored for the development of hair cells using FACS and qRT-PCR. Again, pargyline showed the most potent effect on LCPs, although only when the drug was used in combination with Wnt activation. FACS analysis for Atoh1-nGFP positive cells following differentiation showed that Lsd1 inhibitors alone had no overt effect on hair cell differentiation. However, the addition of pargyline to standard differentiation treatment significantly increased the percentage of Atoh1-nGFP cells in culture (FIG. 1B: 21 and 39%, p<0.05*). Interestingly, no difference in the mRNA levels of Atoh1 was observed in the pargyline-treated cells relative to standard treatment, but Myo7a expression was significantly upregulated (FIGS. 1C and D; p<0.05). It should be noted that in the developing cochlea, Atoh1 and Myo7a are upregulated at E13.5 and 14.5, respectively in nascent hair cells of the basal region of the cochlea (Pan et al., 2012). As development proceeds, Atoh1 expression spreads to the apical hair cells and is subsequently downregulated from E17.5 (Chen et al., 2002). Conversely, Myo7a expression increases throughout development and is a key marker of mature hair cells. All hair cell markers tested were elevated with the exception of Atoh1, which had begun to decrease 10 days after the initiation of differentiation.

These findings suggested that the application of the Lsd1 inhibitor influenced hair cell maturation. This was corroborated by immunostaining, whereby the organoids treated with pargyline were almost uniformly positive for Atoh1 and Myo7a (FIGS. 1G and H). Moreover, the Myo7a+ cells displayed actin-rich protrusions (labeled with phalloidin) emanating from the apical surface, reminiscent of hair cell stereocilia (FIG. 1H). Thus, pargyline was more effective than valproic acid at potentiating the effect of Wnt on hair cell differentiation, implicating Lsd1 as a key regulator in the process of hair cell development.

To confirm that the effect of pharmacologic inhibition of Lsd1 by pargyline and other drugs was Lsd1 specific, we tested the effect of genetic deletion of Lsd1 on hair cell differentiation in the organoid system. We utilized a mouse with a conditional allele of Lsd1, whereby Cre-recombinase mediates excision of exons 5 and 6, generating a frame shift mutation and premature stop. Exons 5 and 6 encode both the flavin adenine dinucleotide binding site and N-terminal portion of the amine oxidase domain, both necessary for the enzymatic activity of Lsd1 (Kerenyi et al., 2013). The Lsd1^{fl/+} mouse was crossed to Sox2-CreER: Atoh1-nGFP mice, to knock out Lsd1 in supporting cells. Heterozygous Lsd1 mice (one deleted allele of Lsd1) were used for this analysis. The organoids generated from these mice were expanded and treated with tamoxifen at the start of differentiation. Negative controls consisted of LCPs generated from littermates without Cre. Consistent with the effect of pargyline, knockdown of Lsd1 potentiated hair cell differentiation based on the proportion of Atoh1-nGFP cells (FIG. 1E: p<0.05*). It also increased the expression of Myo7a, while reducing the total Atoh1 expression at the 10-day time point (FIG. 1F), consistent with the downregulation of Atoh1 during the maturation of as hair cells.

### Example 2. RNA-Sequencing Revealed Increased Expression of Hair Cell Genes after Treatment with Pargyline

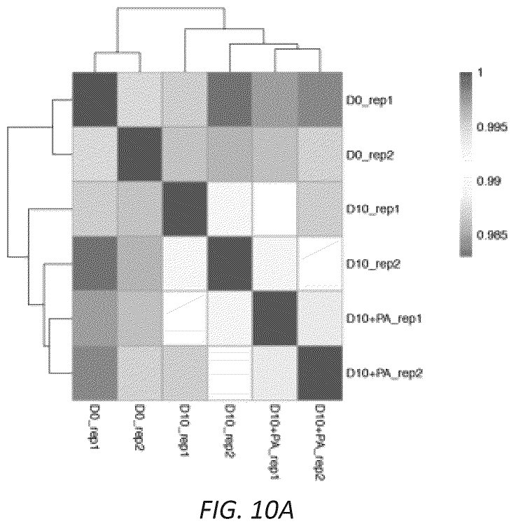

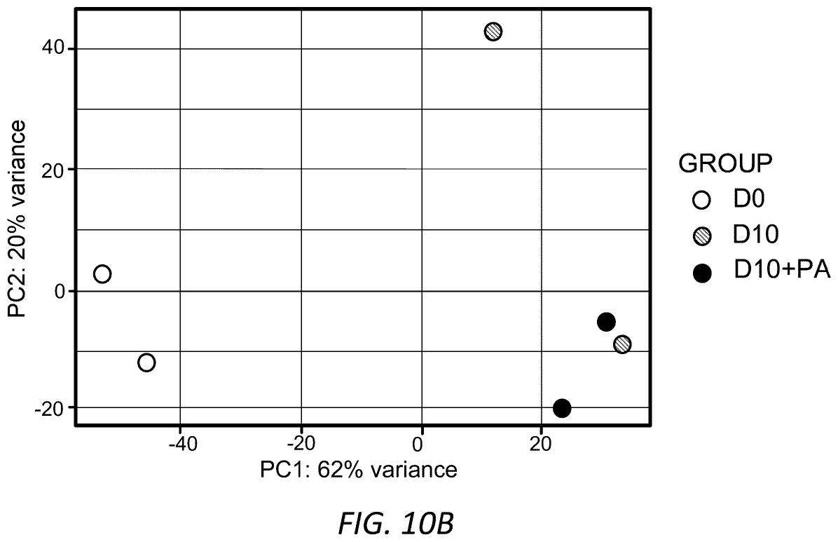

The effect of pargyline on the differentiation of hair cells could be broad and we therefore evaluated its effect on the gene expression patterns of Lsd1 inhibitor-treated and untreated samples. The samples analyzed included the undifferentiated organoids following expansion (DO) and the differentiated organoids following 10 days of differentiation, with and without pargyline treatment (D10). The initial QC analysis of the data revealed that all the samples had 30-65 million reads per sample. In addition, 90% of the genes aligned to the genome and over 22,000 genes were detected in each sample. Pearson correlation heat-map of normalized counts, which was used to visualize the variability between samples, demonstrated clear segregation of samples by the time of differentiation and treatment (0.98-0.99 correlation coefficient: FIG. 10A). The times here refer to the beginning and end of differentiation and the finding was that most of the difference between samples was due to the 10 days of differentiation. Consistently, the principle component analysis (PCA) clustered the samples by the time of differentiation (responsible for 62% of the variation between samples) and the treatment (20% of the variation: FIG. 10B). Lsd1 inhibition in the presence of CHIR99021 altered gene expression and caused the pargyline-treated samples to cluster separately from those treated with CHIR99021 alone.

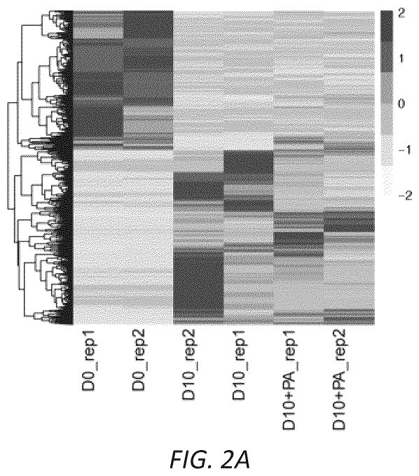

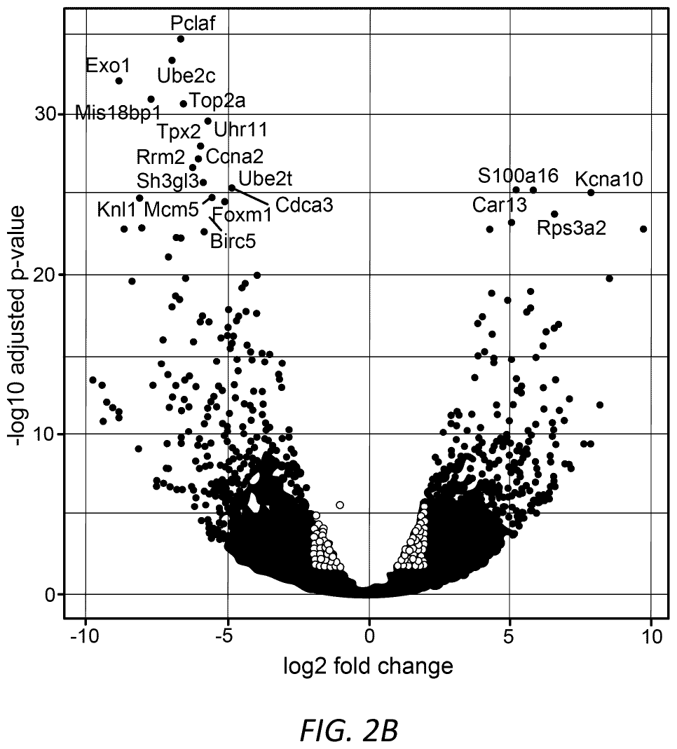

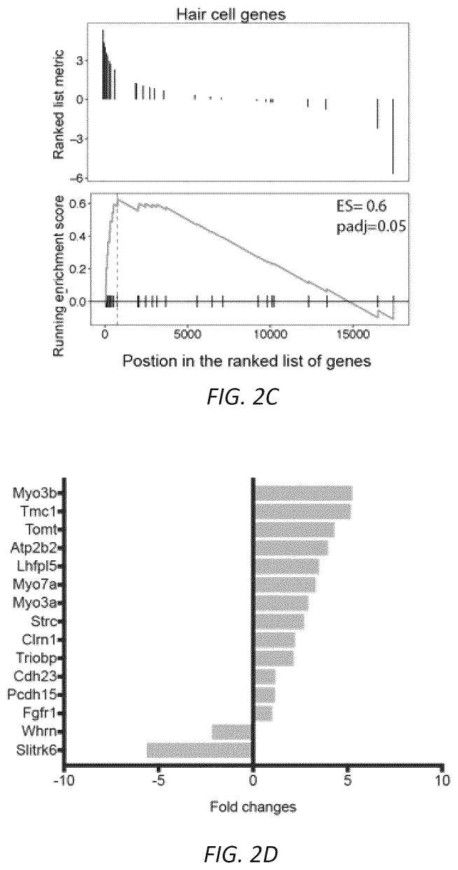

Differential expression analysis of the samples was also performed using DESeq2 (Love et al., 2014). The heat map showing the differentially expressed genes on a per sample basis also showed clear segregation in gene expression in the undifferentiated (DO) versus differentiated samples (D10) (FIG. 2A). We further compared the gene expression profiles of the samples. Compared to the undifferentiated (DO), approximately 2510) and 3365 genes were differentially expressed in the D10 or D10+pargyline samples, respectively. The addition of pargyline compared to the untreated samples altered the expression of approximately 474 genes (padj<0.05, fold change >1.25). Out of these, 144 genes were upregulated, and 330 genes downregulated. The most significantly upregulated genes included Kcna10 and Car13, both expressed in mature cochlear hair cells [FIG. 2B: (Carlisle et al., 2012: Wu et al., 2013)]. Notably, we observed significant downregulation of Ube 2c and Cdca3, genes involved in cell cycle regulation.

Gene set enrichment analysis (GSEA) using gene sets associated with biological process gene ontology terms (Subramanian et al., 2005) was next performed to gain insight into the relevant pathways altered in this dataset. The analysis accounts for coordinated differential expression over pre-defined gene sets, instead of changes in individual genes. Pathways related to the cell cycle and proliferation were the most enriched after pargyline treatment, particularly the p53 and PI3/AKT, MAPK and RAS pathways (−0.503, −0.33). although these cells were in the differentiation phase. Interestingly, a gene set associated with the development of auditory receptors was significantly enriched (score: 0.62, p<0.1: FIG. 2C; GO:0060117). Here we present the top 15 differentially expressed hair cell receptor genes. Specifically, genes including Myo3b, Tme1, Tomt, Atp2b2, Lhfpl5 and Myo7a were upregulated after pargyline treatment (FIG. 2D: p<0.1). Collectively, these findings corroborate increasing hair cell differentiation with Lsd1 inhibition.

### Example 3. The Activity of the Wnt/β-Catenin Pathway was not Significantly Altered after Treatment with Pargyline

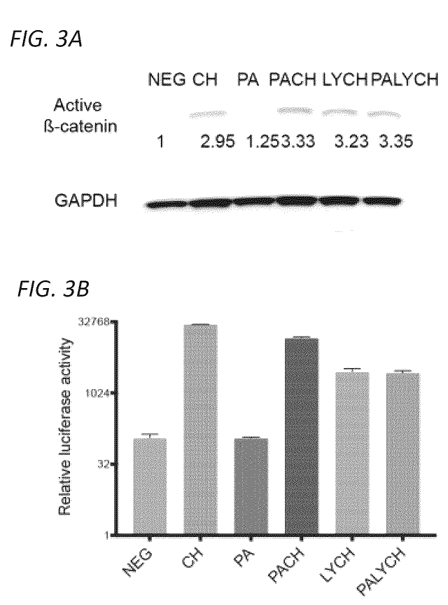

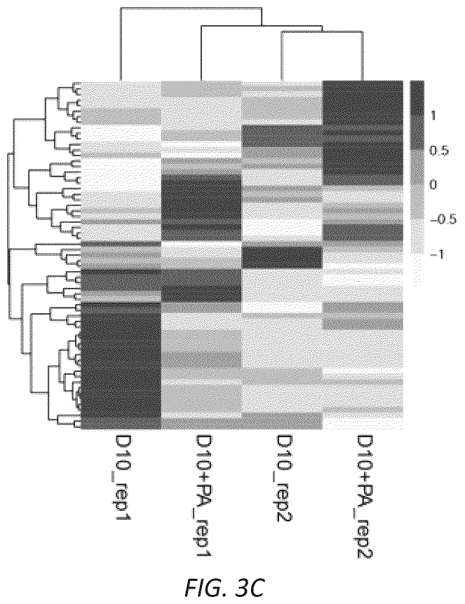

Active canonical Wnt signaling is facilitated by the nuclear translocation of β-catenin, which binds with transcription factors of the Tcf/Lef family to activate transcription. To further validate our RNA-sequencing data, we proceeded to examine the levels of active β-catenin using western blot after pargyline treatment. Here, we observed some accumulation of active β-catenin after CHIR99021 or LYCH treatment (fold changes: 2.95 and 3.23). With the addition of pargyline, the levels of active β-catenin were increased, but marginally (fold changes-PACH-3.33, PALYCH-3.35; FIG. 3A). We also analyzed Wnt activity in the treated cells using a Tcf/Lef reporter assay. Here, we observed an increase in Tcf/Lef activity after treatment with CHIR99021 and LYCH, but with the addition of pargyline, no significant difference was observed in Tcf/Lef activity between the treatments (FIG. 3B). Collectively, these findings suggest that effect of pargyline is not mediated by activation of Wnt target genes.

We next focused on elucidating the mechanism underlying the activity of pargyline on hair cell differentiation. We previously demonstrated that Atoh1 is a key Wnt target gene, as evidenced by its expression being modulated by the level of β-catenin bound to its regulatory chromatin domains (Shi et al., 2010). As a role for Lsd1 in regulating the expression of Wnt target genes has previously been established (Chen et al., 2016; Lei et al., 2015: Zhou et al., 2016), we first asked if the effect of pargyline on hair cell differentiation was mediated by changes in activity of other Wnt target genes. We curated a list of genes using previously published data to examine the differential expression of Wnt target genes in LCPs treated with pargyline (Hodar et al., 2010; Nusse, 2018; Railo et al., 2009; Watanabe et al., 2014). The heatmap depicts the differential expression pattern of all the Wnt target genes (FIG. 3C). Here, it is evident that there is some variability between the replicates, but the activated Wnt target genes in the pargyline-treated versus untreated samples showed a clear discrepancy. Upon examination of the top 15 differentially expressed Wnt target genes, 13 genes such as Cond1, Jag1 and Hes1 were downregulated, while only 2 genes Mmp14 and Sgk were upregulated in the pargyline-treated samples (p<0).05, FIG. 3D).

### Example 4. Atoh1 Transcription was Suppressed by Targeting a Nuclease Deficient dCas-9 Lsd1 to the Promoter or Enhancer Regions of the Atoh1 Gene

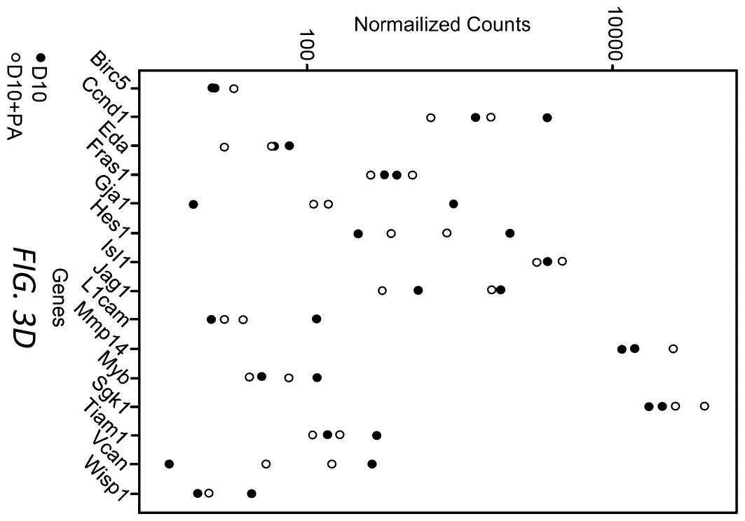

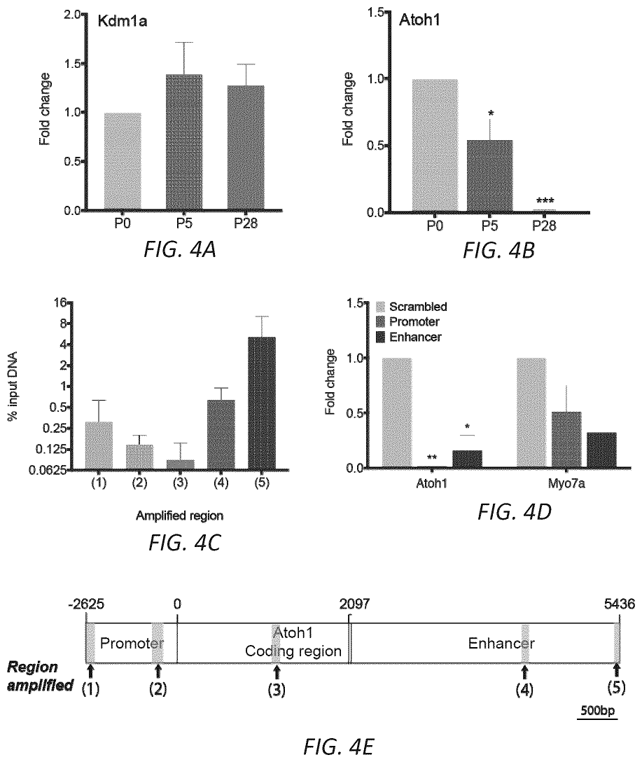

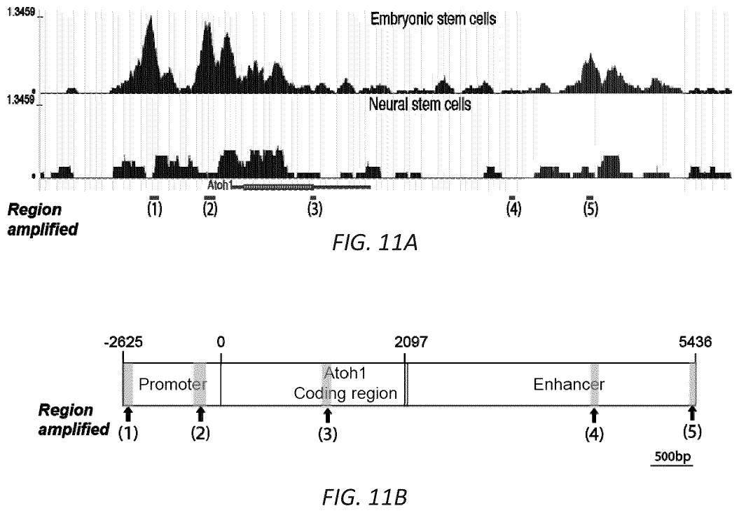

We next focused on the effects of Lsd1 on Atoh1 expression. Given the effect of Lsd1 on LCPs in inducing their differentiation into hair cells, we examined the expression pattern of Lsd1 and Atoh1 in the developing and mature cochlea. The qRT-PCR data revealed that Lsd1 was expressed in the cochlea and its expression was maintained postnatally from PO to adult (P28: FIG. 4A). FIG. 4 shows that Lsd1 expression goes up in the cochlea as Atoh1 expression decreases. (FIG. 4B: p<0.01* and <0.001***). The persistent expression of Lsd1 in the cochlea implicates that it may play a regulatory role in hair cell differentiation. We used the LCP-derived hair cells to examine the specific sites that Lsd1 occupies on the Atoh1 locus using ChIP-qPCR. We found that Lsd1 occupies the promoter (region 1) and enhancer (region 5) of the Atoh1 gene during hair cell differentiation (FIG. 4C). These findings were consistent with prior ChIP-seq analyses of ESCs and neural stem cells, whereby Lsd1 was observed to occupy the promoter and enhancer regions (FIG. 11A: (Wang et al., 2016: Whyte et al., 2012)]. Interestingly, in ESCs, the density of Lsd1 signals was higher in the promoter regions compared to the enhancer, whereas in neural stem cells, the signals at the promoter and enhancer regions were comparable. Collectively, this data suggests that Lsd1 has a significant role in regulating Atoh1 expression.

We next generated a nuclease-deficient Cas9 (dCas9)-fused to Lsd1 and used a viral delivery system for single guide RNAs (Kearns et al., 2015). We used this system to target the promoter 2 and enhancer regions 2 of the Atoh1 gene. The cells were transduced with the virus at the start of proliferation and differentiated using our previously published protocol (McLean et al., 2017). At the end of differentiation, the transduced cells were collected using FACS and analyzed for hair cell markers using qRT-PCR. Targeting of Lsd1 to both the promoter and enhancer regions of Atoh1 resulted in a significant loss of Atoh1 expression (FIG. 4D: p<0.05* and p<0.01**). In addition, expression of Myo7a was also reduced in the dCas9-Lsd1 transduced cells. Differentiation was unaffected in the cells transduced with the scrambled gRNA. Overall, this data implicates the regulatory effect of Lsd1 on Atoh1 transcription.

### Example 5. Chromatin Immunoprecipitation Revealed that Atoh1 Upregulation was Concurrent with the Accumulation of H3K4Me and H3K4Me2 at the Promoter and Enhancer Regions of Atoh1

In order to further dissect the mechanism of Lsd1 activity, we analyzed changes in epigenetic marks on several sites along the Atoh1 gene using chromatin immunoprecipitation. We used the LCPs starting at day 0 (D0) of differentiation with day 10 (D10) as the endpoint (FIG. 5A). Lsd1 has been characterized to demethylate the activating epigenetic marks H3K4me and H3K4me2 (Shi et al., 2004). Previous studies have revealed that the H3K4me2 modification is enriched particularly in promoters, at transcriptionally active genes or genes primed for future expression during cell development (Bernstein et al., 2002; Koch et al., 2007: Orford et al., 2008). Consistently, we observed accumulation of the activating H3K4me2 mark at the proximal promoter region (region 2) of the Atoh1 gene during hair cell differentiation (D10, p<0.05*: FIG. 5B). With the addition of pargyline, the level of H3K4me2 was significantly increased (p<0.01**) compared to the untreated (DO). The levels of H3K4me2 were also increased at the enhancer regions (region 5), but the changes were not statistically significant. In addition, we examined the enrichment levels of H3K4me around the Atoh1 locus. We found significant enrichment of the H3K4me mark at the enhancer region after pargyline treatment (region 5, p<0.01**; FIG. 5C). H3K4me has previously been identified to be a marker of active enhancers, thus suggesting that pargyline may have a role in increasing activation of the Atoh1 enhancer (Heintzman et al., 2007). Collectively, these findings reveal that Lsd1 inhibition increases accumulation of activating epigenetic marks, thereby facilitating the opening of the chromatin and activating transcription of the Atoh1 gene.

As Lsd1 is also known to demethylate H3K9me2, we analyzed the changes in the levels of this repressive mark at the Atoh1 locus (Metzger et al., 2005). The levels of H3K9me2 on the Atoh1 gene were not significantly altered in the undifferentiated and differentiated treatments (FIG. 5D). Addition of pargyline did not significantly alter enrichment for H3K9me2: the level of H3K9me2 were reduced at the promoter (region 2) and enhancer regions (region 5), but the difference was not significant.

### Example 6. Lsd1 Inhibition Induced Hair Cell Differentiation in Neonatal Cochlear Explants

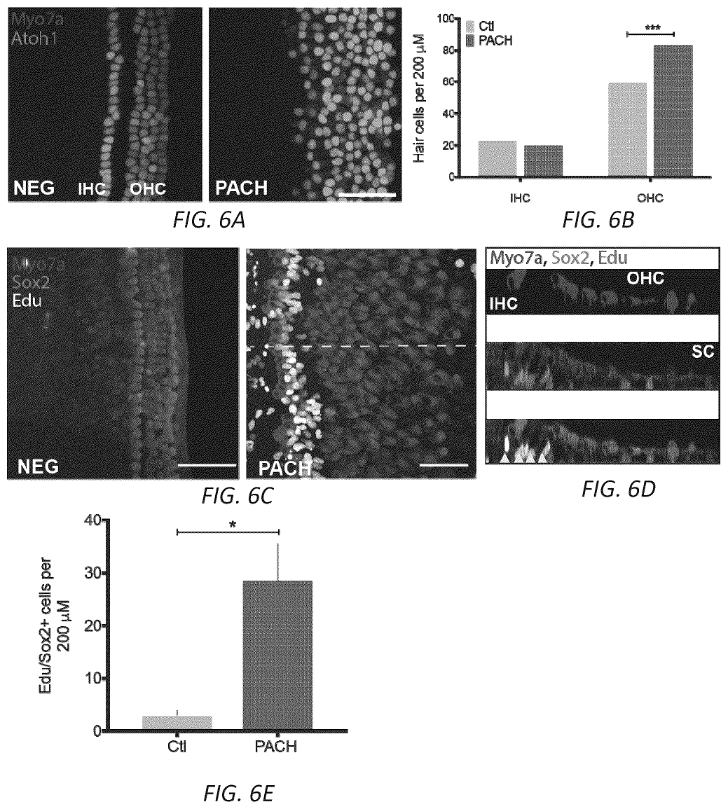

Having established that Lsd1 inhibition promotes differentiation of hair cells in our organoid model through epigenetic mechanism by altering H3K4Me2 markss, we looked at a potential for Lsd1 inhibition to induce hair cell differentiation in new born cochlear tissue derived from Atoh1-nGFP mice. Newborn organ of Corti explants were cultured in specific drug combinations for up to 3 days. The addition of pargyline combined with CHI99021 increased the number of Atoh1-GFP and Myo7a-positive cells in the outer hair cell region. While no significant difference was observed in the number of inner hair cells, the number of outer hair cells in the middle region of the cochlea was significantly increased to 84 cells/200 μm compared to the untreated 59 cells/200 μm (p<0.001***FIGS. 6A and D, p<0.01), further supporting our findings that Lsd1 inhibition enhances hair cell differentiation in the neonatal cochlea.

Wnt activation by overexpressing β-catenin (Chai et al., 2012; Shi et al., 2013; Shi et al., 2012) or applying a GSK3β inhibitor such as CHIR99021 (McLean et al., 2017) can induce proliferation of inner ear progenitor cells. To assay the effect of Lsd1 on supporting cell proliferation, we assayed for EdU incorporation in organ of Corti explants treated with pargyline and CHIR99021 for 3 days. We found significant incorporation of EdU in supporting cells throughout the cochlea (FIGS. 6B and C). A significant increase in the number of Edu+/Sox2+ (28 cells per 200 μm) was observed in the pargyline and CHIR99021-treated explants. Interestingly, supporting cell proliferation was most prominent in the Lgr5+ inner border cells. Consistent with our LCP data (FIG. 1A), we found a significant induction in proliferation of neonatal supporting cells after treatment with pargyline.

### Example 7. Partial Hearing Recovery and Transdifferentiation of Supporting Cells into Hair Cells after Pargyline and CHIR99021 Treatment of the Deaf Adult Cochlea

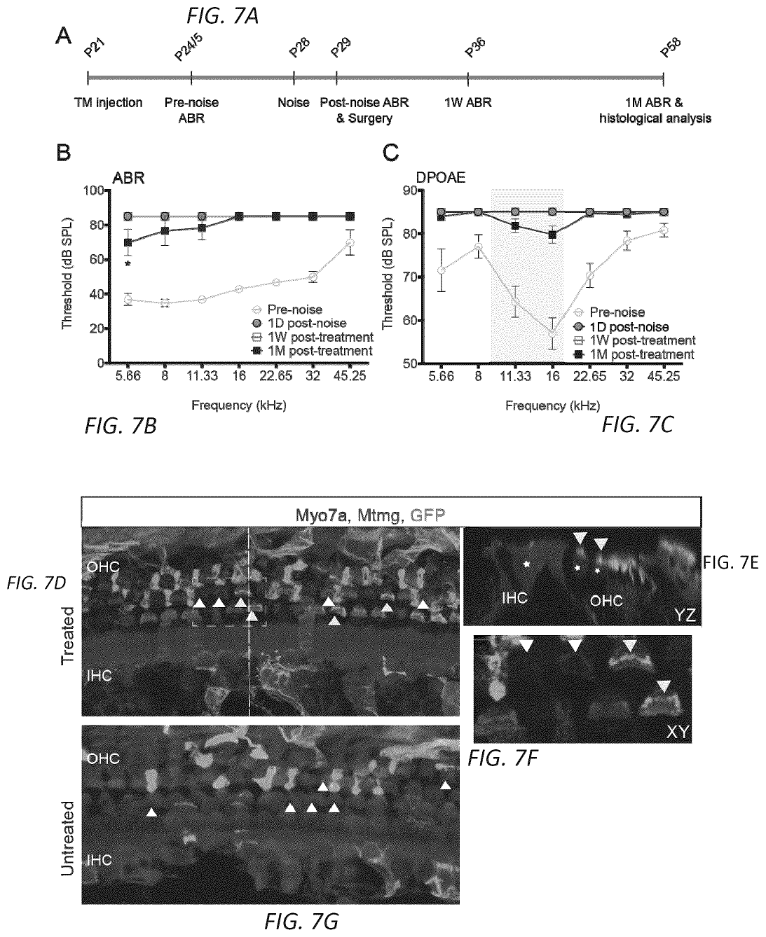

In the adult cochlea, supporting cells are quiescent and have restricted differentiation potentials. We previously reported the potential to promote hair cell regeneration and hearing restoration in the noise-damaged adult cochlea after treatment with the Notch inhibitor LY411575 (Mizutari et al., 2013). Here, we analyzed the effect of Lsd1 inhibition on the noise-damaged adult cochlea. Four-week-old mice were exposed to noise and treated with drug the next day via local administration at the round window membrane. The ABRs were analyzed 1 day, 1 week and 1-month post-treatment (FIG. 7A). ABR and DPOAE thresholds 1 day after noise exposure were >80 dB sound pressure level (SPL) at all frequencies (FIGS. 7B and C). Threshold changes were not observed in the untreated contralateral ears. After treatment with pargyline and CHIR99021, no ABR threshold changes were observed after 1 week (FIG. 7B). However, after 1 month, threshold recoveries of ≤25-30 dB SPL were observed at 5.66, 8 and 11.33 kHz in 2 of the 5 animals analyzed (FIG. 7B, p<0.05*, n=5). No threshold recoveries were observed in either ear at frequencies above 16 kHz by ABR and no recoveries above the noise floor of DPOAE could be detected.

We also used in vivo lineage tracing to test whether new hair cells were generated after the treatment. As previously reported, we used a Cre-reporter strain to perform lineage tracing of Sox2-positive cells, as Sox2 is expressed in supporting cells (Bramhall et al., 2014). We generated Sox2-CreER: mT/mG mice, where the cells expressing Sox2 at the time of tamoxifen administration become permanently tagged for green fluorescent protein (GFP) (Fujioka et al., 2015; Mizutari et al., 2013). One month after pargyline and CHIR99021 treatment, we observed some Myo7a-positive cells in the deafened cochlea that expressed GFP, demonstrating transdifferentiation from Sox2-positive cells. Notably, we observed green hair bundles in the myosin VIIa/GFP double-labeled cells, and some of the bundles had a V-shaped structure, reminiscent of original hair cells (FIG. 7E-XY and YZ planes). The new hair cells were predominantly observed in the apical-mid regions of the cochlea. These results suggested that pargyline and CHIR99021 treatment induced supporting cell transdifferentiation into hair cells and also restored some auditory function.

### Example 8. Changes in Gene Expression of Wnt Target Genes after Noise Damage and Treatment with Pargyline and CHIR99021

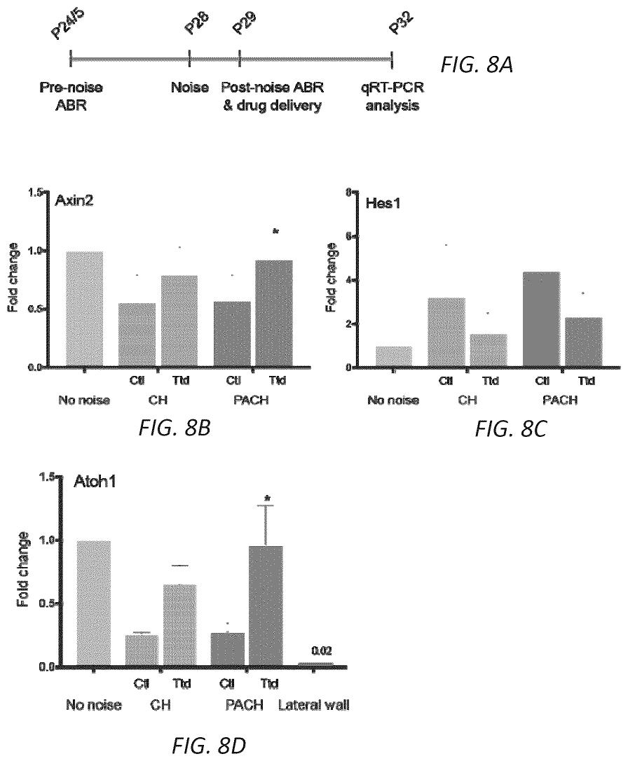

While accumulating evidence indicates the necessity of the Wnt pathway in hair cell differentiation, the adult cochlea is not responsive to Wnt activation (Chai et al., 2012: Shi et al., 2013; Shi et al., 2012). Given our evidence that pargyline increases the levels of H3K4me/2 at the Atoh1 promoter and activates Atoh1 expression in the new born cochlea we wanted to whether the mechanism of Lsd1 inhibition relied on the action of the Wnt pathway in the adult as it did in the newborn, We next wanted to determine if Wnt activity was maintained in adults. In Wnt reporter mice, which express an H2B-EGFP fusion protein under the control a Tcf/Lef response element and heat shock protein 1B minimal promoter to visualize Wnt activity (Ferrer-Vaquer et al., 2010), we found that the Wnt pathway remained active in the adult cochlea, specifically in inner hair cells, Claudius and Hensen cells (FIG. 8A).

We used qRT-PCR to examine gene expression in the adult mouse cochlea after noise damage with or without treatment with pargyline and CHIR99021 (FIG. 8A). We first assessed the mRNA changes in the Wnt target gene, Axin2, and Notch effector gene, Hes1, in the deafened mature cochlea after treatment with pargyline and CHIR99021. Three days after the noise exposure, Axin2 expression was downregulated (FIG. 8B), while Hes1 mRNA increased (FIG. 8C), compared to their pre-noise levels. Upon administration of CHIR99021 alone or in combination with or pargyline, these effects were reversed: the treated-cochlea had significantly higher levels of Axin2 (p<0.05*) and reduced levels of Hes1. Axin2 in the pargyline and CHIR99021-treated cochlea was increased. Consistent with previous findings (Mizutari et al., 2013), there was a decline in Atoh1 upon noise exposure (FIGS. 8D, 8E). However, treatment with CHIR99021 or pargyline and CHIR99021, resulted in significantly elevated Atoh1 in the deaf adult cochlea relative to the control (p<0.05*) 3 days after treatment. Collectively, these data demonstrated that Lsd1 inhibition could activate expression of Atoh1 in the deaf adult cochlea. Based on the epigenetic effects seen in LCPs, the increased expression of Atoh1, which is not normally expressed in the adult cochlea, could be caused by a similar mechanism after Lsd1 inhibition in the adult cochlea.

At 4 days after noise exposure the Wnt reporter shows activity in the supporting cells and hair cells. This suggests that the Wnt pathway is still active at this age. Expression levels of the Tcf/Lef reporter determined by RT-PCR were increased by the drug treatment, confirming the PCR results for Axin1.

To further confirm our Atoh1 qPCR data, we treated Atoh1-GFP mouse (Zoghbi—provide reference) in which Atoh1 is linked to GFP. This is distinct from the Atoh1-nGFP mouse which was used throughout the paper which is comprised of Atoh1—Enhancer transgene GFP reporter. Using the Zoghbi mouse, we identified Atoh1-GFP+ stained cells following treatment of the adult cochlea with pargyline and CHIR99021. Expression in the cytoplasm is likely due to newly made Atoh1 as suggested in previous work on this mouse and by examination of a new born mouse where Atoh1 expression was seen at in the nucleus but also in the cytoplasm after stimulation of Atoh1 by treatment with pargyline and CHIR99021.

### REFERENCES

Adamo, A., Sese, B., Boue, S., Castano, J., Paramonov, I., Barrero, M. J., and Izpisua Belmonte, J. C. (2011). LSD1 regulates the balance between self-renewal and differentiation in human embryonic stem cells. Nat Cell Biol 13, 652-659.
Ahmed, M., and Streit, A. (2018). Lsd1 interacts with cMyb to demethylate repressive histone marks and maintain inner ear progenitor identity. Development 145.
Azuara, V., Perry, P., Sauer, S., Spivakov, M., Jorgensen, H. F., John, R. M., Gouti, M., Casanova, M., Warnes, G., Merkenschlager, M., et al. (2006). Chromatin signatures of pluripotent cell lines. Nat Cell Biol 8, 532-538.
Ballas, N., Battaglioli, E., Atouf, F., Andres, M. E., Chenoweth, J., Anderson, M. E., Burger, C., Moniwa, M., Davie, J. R., Bowers, W. J., et al. (2001). Regulation of neuronal traits by a novel transcriptional complex. Neuron 31, 353-365.
Barker, N., van Es, J. H., Kuipers, J., Kujala, P., van den Born, M., Cozijnsen, M., Haegebarth, A., Korving, J., Begthel, H., Peters, P. J., et al. (2007). Identification of stem cells in small intestine and colon by marker gene Lgr5. Nature 449, 1003-1007.
Bermingham, N. A., Hassan, B. A., Price, S. D., Vollrath, M. A., Ben-Arie, N., Eatock, R. A., Bellen, H. J., Lysakowski, A., and Zoghbi, H. Y. (1999). Math 1: an essential gene for the generation of inner ear hair cells. Science 284, 1837-1841.
Bernstein, B. E., Humphrey, E. L., Erlich, R. L., Schneider, R., Bouman, P., Liu, J. S., Kouzarides, T., and Schreiber, S. L. (2002). Methylation of histone H3 Lys 4 in coding regions of active genes. Proc Natl Acad Sci USA 99, 8695-8700.
Bernstein, B. E., Mikkelsen, T. S., Xie, X., Kamal, M., Huebert, D. J., Cuff, J., Fry, B., Meissner, A., Wernig, M., Plath, K., et al. (2006). A bivalent chromatin structure marks key developmental genes in embryonic stem cells. Cell 125, 315-326.
Bramhall, N. F., Shi, F., Arnold, K., Hochedlinger, K., and Edge, A. S. (2014). Lgr5-positive supporting cells generate new hair cells in the postnatal cochlea. Stem Cell Reports 2, 311-322.
Cai, T., Jen, H. I., Kang, H., Klisch, T. J., Zoghbi, H. Y., and Groves, A. K. (2015). Characterization of the transcriptome of nascent hair cells and identification of direct targets of the Atoh1 transcription factor. J Neurosci 35, 5870-5883.
Cai, T., Seymour, M. L., Zhang, H., Pereira, F. A., and Groves, A. K. (2013). Conditional deletion of Atoh1 reveals distinct critical periods for survival and function of hair cells in the organ of Corti. J Neurosci 33, 10110-10122.
Carlisle, F. A., Steel, K. P., and Lewis, M. A. (2012). Specific expression of Kcna10, Pxn and Odf2 in the organ of Corti. Gene Expr Patterns 12, 172-179.
Chai, R., Kuo, B., Wang, T., Liaw, E. J., Xia, A., Jan, T. A., Liu, Z., Taketo, M. M., Oghalai, J. S., Nusse, R., et al. (2012). Wnt signaling induces proliferation of sensory precursors in the postnatal mouse cochlea. Proc Natl Acad Sci USA 109, 8167-8172.
Chen, P., Johnson, J. E., Zoghbi, H. Y., and Segil, N. (2002). The role of Math 1 in inner ear development: Uncoupling the establishment of the sensory primordium from hair cell fate determination. Development 129, 2495-2505.
Chen, P., and Segil, N. (1999). p27(Kip1) links cell proliferation to morphogenesis in the developing organ of Corti. Development 126, 1581-1590.
Chen, Y., Kim, J., Zhang, R., Yang, X., Zhang, Y., Fang, J., Chen, Z., Teng, L., Chen, X., Ge, H., et al. (2016). Histone Demethylase LSD1 Promotes Adipocyte Differentiation through Repressing Wnt Signaling. Cell Chem Biol 23, 1228-1240.
Chonko, K. T., Jahan, I., Stone, J., Wright, M. C., Fujiyama, T., Hoshino, M., Fritzsch, B., and Maricich, S. M. (2013). Atoh1 directs hair cell differentiation and sur vival in the late embryonic mouse inner ear. Dev Biol 381, 401-410.
Cox, B. C., Chai, R., Lenoir, A., Liu, Z., Zhang, L., Nguyen, D. H., Chalasani, K., Steigelman, K. A., Fang, J., Rubel, E. W., et al. (2014). Spontaneous hair cell regeneration in the neonatal mouse cochlea in vivo. Development 141, 816-829.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics (Oxford, England) 29, 15-21.
Ferrer-Vaquer, A., Piliszek, A., Tian, G., Aho, R. J., Dufort, D., and Hadjantonakis, A. K. (2010). A sensitive and bright single-cell resolution live imaging reporter of Wnt/ss-catenin signaling in the mouse. BMC Dev Biol 10, 121.
Forneris, F., Binda, C., Vanoni, M. A., Mattevi, A., and Battaglioli, E. (2005). Histone demethylation catalysed by LSD1 is a flavin-dependent oxidative process. FEBS Lett 579, 2203-2207.
Fuerer, C., and Nusse, R. (2010). Lentiviral vectors to probe and manipulate the Wnt signaling pathway. PLOS One 5, e9370.
Fujioka, M., Okano, H., and Edge, A. S. (2015). Manipulating cell fate in the cochlea: a feasible therapy for hearing loss. Trends Neurosci 38, 139-144.
Geng, R., Noda, T., Mulvaney, J. F., Lin, V. Y., Edge, A. S., and Dabdoub, A. (2016). Comprehensive Expression of Wnt Signaling Pathway Genes during Development and Maturation of the Mouse Cochlea. PLOS One 11, e0148339.
Groves, A. K. (2010). The challenge of hair cell regeneration. Exp Biol Med (Maywood) 235, 434-446.
Hanna, J. H., Saha, K., and Jaenisch, R. (2010). Pluripotency and cellular reprogramming: facts, hypotheses, unresolved issues. Cell 143, 508-525.
Heintzman, N. D., Stuart, R. K., Hon, G., Fu, Y., Ching, C. W., Hawkins, R. D., Barrera, L. O., Van Calcar, S., Qu, C., Ching, K. A., et al. (2007). Distinct and predictive chromatin signatures of transcriptional promoters and enhancers in the human genome. Nat Genet 39, 311-318.
Hodar, C., Assar, R., Colombres, M., Aravena, A., Pavez, L., Gonzalez, M., Martinez, S., Inestrosa, N. C., and Maass, A. (2010). Genome-wide identification of new Wnt/beta-catenin target genes in the human genome using CART method. BMC Genomics 11, 348.
Hu, L., Lu, J., Chiang, H., Wu, H., Edge, A. S., and Shi, F. (2016). Diphtheria Toxin-Induced Cell Death Triggers Wnt-Dependent Hair Cell Regeneration in Neonatal Mice. J Neurosci 36, 9479-9489.
Hu, M., Kurobe, M., Jeong, Y. J., Fuerer, C., Ghole, S., Nusse, R., and Sylvester, K. G. (2007). Wnt/beta-catenin signaling in murine hepatic transit amplifying progenitor cells. Gastroenterology 133, 1579-1591.
Jacques, B. E., Puligilla, C., Weichert, R. M., Ferrer-Vaquer, A., Hadjantonakis, A. K., Kelley, M. W., and Dabdoub, A. (2012). A dual function for canonical Wnt/beta-catenin signaling in the developing mammalian cochlea. Development 139, 4395-4404.
Jaenisch, R., and Bird, A. (2003). Epigenetic regulation of gene expression: how the genome integrates intrinsic and environmental signals. Nat Genet 33 Suppl, 245-254.
Jorstad, N. L., Wilken, M. S., Grimes, W. N., Wohl, S. G., VandenBosch, L. S., Yoshimatsu, T., Wong, R. O., Rieke, F., and Reh, T. A. (2017). Stimulation of functional neuronal regeneration from Muller glia in adult mice. Nature 548, 103-107.
Kearns, N. A., Pham, H., Tabak, B., Genga, R. M., Silverstein, N. J., Garber, M., and Maehr, R. (2015). Functional annotation of native enhancers with a Cas9-histone demethylase fusion. Nat Methods 12, 401-403.
Kerenyi, M. A., Shao, Z., Hsu, Y. J., Guo, G., Luc, S., O'Brien, K., Fujiwara, Y., Peng, C., Nguyen, M., and Orkin, S. H. (2013). Histone demethylase Lsd1 represses hematopoietic stem and progenitor cell signatures during blood cell maturation. Elife 2, e00633.
Koch, C. M., Andrews, R. M., Flicek, P., Dillon, S. C., Karaoz, U., Clelland, G. K., Wilcox, S., Beare, D. M., Fowler, J. C., Couttet, P., et al. (2007). The landscape of histone modifications across 1% of the human genome in five human cell lines. Genome Res 17, 691-707.
Laurent, B., Ruitu, L., Murn, J., Hempel, K., Ferrao, R., Xiang, Y., Liu, S., Garcia, B. A., Wu, H., Wu, F., et al. (2015). A specific LSD1/KDMIA isoform regulates neuronal differentiation through H3K9 demethylation. Mol Cell 57, 957-970.
Lei, Z. J., Wang, J., Xiao, H. L., Guo, Y., Wang, T., Li, Q., Liu, L., Luo, X., Fan, L. L., Lin, L., et al. (2015). Lysine-specific demethylase 1 promotes the stemness and chemoresistance of Lgr5+ liver cancer initiating cells by suppressing negative regulators of beta-catenin signaling. Oncogene 34, 3214.
Love, M. I., Huber, W., and Anders, S. (2014). Moderated estimation of fold change and dispersion for RNA-seq data with DESeq2. Genome Biol 15, 550.
Lumpkin, E. A., Collisson, T., Parab, P., Omer-Abdalla, A., Haeberle, H., Chen, P., Doetzlhofer, A., White, P., Groves, A., Segil, N., et al. (2003). Math1-driven GFP expression in the developing nervous system of transgenic mice. Gene Expr Patterns 3, 389-395.
Maiques-Diaz, A., Spencer, G. J., Lynch, J. T., Ciceri, F., Williams, E. L., Amaral, F. M. R., Wiseman, D. H., Harris, W. J., Li, Y., Sahoo, S., et al. (2018). Enhancer Activation by Pharmacologic Displacement of LSD1 from GFIl Induces Differentiation in Acute Myeloid Leukemia. Cell Rep 22, 3641-3659.
Maru, Y., Orihashi, K., and Hippo, Y. (2016). Lentivirus-Based Stable Gene Delivery into Intestinal Organoids. Methods Mol Biol 1422, 13-21.
McLean, W. J., Yin, X., Lu, L., Lenz, D. R., McLean, D., Langer, R., Karp, J. M., and Edge, A. S. (2017). Clonal Expansion of Lgr5-Positive Cells from Mammalian Cochlea and High-Purity Generation of Sensory Hair Cells. Cell Rep 18, 1917-1929.
Metzger, E., Wissmann, M., Yin, N., Muller, J. M., Schneider, R., Peters, A. H., Gunther, T., Buettner, R., and Schule, R. (2005). LSD1 demethylates repressive histone marks to promote androgen-receptor-dependent transcription. Nature 437, 436-439.
Mizutari, K., Fujioka, M., Hosoya, M., Bramhall, N., Okano, H. J., Okano, H., and Edge, A. S. (2013). Notch inhibition induces cochlear hair cell regeneration and recovery of hearing after acoustic trauma. Neuron 77, 58-69.
Nusse, R. (2018). Wnt Target genes (web.stanford.edu/group/nusselab/cgi-bin/wnt/target_genes).
Ocampo, A., Reddy, P., Martinez-Redondo, P., Platero-Luengo, A., Hatanaka, F., Hishida, T., Li, M., Lam, D., Kurita, M., Beyret, E., et al. (2016). In Vivo Amelioration of Age-Associated Hallmarks by Partial Reprogramming. Cell 167, 1719-1733 e1712.
Orford, K., Kharchenko, P., Lai, W., Dao, M. C., Worhunsky, D. J., Ferro, A., Janzen, V., Park, P. J., and Scadden, D. T. (2008). Differential H3K4 methylation identifies developmentally poised hematopoietic genes. Dev Cell 14, 798-809.
Pan, N., Kopecky, B., Jahan, I., and Fritzsch, B. (2012). Understanding the evolution and development of neurosensory transcription factors of the ear to enhance therapeutic translation. Cell Tissue Res 349, 415-432.
Park, W. Y., Hong, B. J., Lee, J., Choi, C., and Kim, M. Y. (2016). H3K27 Demethylase JMJD3 Employs the NF-kappaB and BMP Signaling Pathways to Modulate the Tumor Microenvironment and Promote Melanoma Progression and Metastasis. Cancer Res 76, 161-170.
Patel, D., Shimomura, A., Majumdar, S., Holley, M. C., and Hashino, E. (2018). The histone demethylase LSD1 regulates inner ear progenitor differentiation through interactions with Pax2 and the NuRD repressor complex. PLOS One 13, e0191689.
Railo, A., Pajunen, A., Itaranta, P., Naillat, F., Vuoristo, J., Kilpelainen, P., and Vainio, S. (2009). Genomic response to Wnt signalling is highly context-dependent—evidence from DNA microarray and chromatin immunoprecipitation screens of Wnt/TCF targets. Exp Cell Res 315, 2690-2704.
Samarajeewa, A., Lenz, D. R., Xie, L., Chiang, H., Kirchner, R., Mulvaney, J. F., Edge, A. S. B., and Dabdoub, A. (2018). Transcriptional response to Wnt activation regulates the regenerative capacity of the mammalian cochlea. Development In press.
Shi, F., Cheng, Y. F., Wang, X. L., and Edge, A. S. (2010). Beta-catenin up-regulates Atoh1 expression in neural progenitor cells by interaction with an Atoh1 3′ enhancer. J Biol Chem 285, 392-400.
Shi, F., Hu, L., and Edge, A. S. (2013). Generation of hair cells in neonatal mice by beta-catenin overexpression in Lgr5-positive cochlear progenitors. Proc Natl Acad Sci USA 110, 13851-13856.
Shi, F., Hu, L., Jacques, B. E., Mulvaney, J. F., Dabdoub, A., and Edge, A. S. (2014). beta-Catenin is required for hair-cell differentiation in the cochlea. J Neurosci 34, 6470-6479.
Shi, F., Kempfle, J. S., and Edge, A. S. (2012). Wnt-responsive lgr5-expressing stem cells are hair cell progenitors in the cochlea. J Neurosci 32, 9639-9648.
Shi, Y., Lan, F., Matson, C., Mulligan, P., Whetstine, J. R., Cole, P. A., Casero, R. A., and Shi, Y. (2004). Histone demethylation mediated by the nuclear amine oxidase homolog LSD1. Cell 119, 941-953.
Stojanova, Z. P., Kwan, T., and Segil, N. (2015). Epigenetic regulation of Atoh1 guides hair cell development in the mammalian cochlea. Development 142, 3529-3536.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc Natl Acad Sci USA 102, 15545-15550.
Sun, G., Alzayady, K., Stewart, R., Ye, P., Yang, S., Li, W., and Shi, Y. (2010). Histone demethylase LSD1 regulates neural stem cell proliferation. Mol Cell Biol 30, 1997-2005.
Wang, Y., Wu, Q., Yang, P., Wang, C., Liu, J., Ding, W., Liu, W., Bai, Y., Yang, Y., Wang, H., et al. (2016). LSD1 co-repressor Rcor2 orchestrates neurogenesis in the developing mouse brain. Nat Commun 7, 10481.
Watanabe, S., Arimura, Y., Nagaishi, K., Isshiki, H., Onodera, K., Nasuno, M., Yamashita, K., Idogawa, M., Naishiro, Y., Murata, M., et al. (2014). Conditioned mesenchymal stem cells produce pleiotropic gut trophic factors. J Gastroenterol 49, 270-282.
Whyte, W. A., Bilodeau, S., Orlando, D. A., Hoke, H. A., Frampton, G. M., Foster, C. T., Cowley, S. M., and Young, R. A. (2012). Enhancer decommissioning by LSD1 during embryonic stem cell differentiation. Nature 482, 221-225.
Wu, L., Sagong, B., Choi, J. Y., Kim, U. K., and Bok, J. (2013). A systematic survey of carbonic anhydrase mRNA expression during mammalian inner ear development. Dev Dyn 242, 269-280.
Yamada, Y., Aoki, H., Kunisada, T., and Hara, A. (2010). Rest promotes the early differentiation of mouse ESCs but is not required for their maintenance. Cell Stem Cell 6, 10-15.
Zhou, A., Lin, K., Zhang, S., Chen, Y., Zhang, N., Xue, J., Wang, Z., Aldape, K. D., Xie, K., Woodgett, J. R., et al. (2016). Nuclear GSK3beta promotes tumorigenesis by phosphorylating KDMIA and inducing its deubiquitylation by USP22. Nat Cell Biol 18, 954-966.
Zibetti, C., Adamo, A., Binda, C., Forneris, F., Toffolo, E., Verpelli, C., Ginelli, E., Mattevi, A., Sala, C., and Battaglioli, E. (2010). Alternative splicing of the histone demethylase LSD1/KDMI contributes to the modulation of neurite morphogenesis in the mammalian nervous system. J Neurosci 30, 2521-2532.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating sensorineural hearing loss associated with loss of auditory hair cells or balance loss associated with a loss of vestibular hair cells in a subject, the method comprising administering to the subject:
(i) a pharmaceutical composition comprising a Histone Lysine Demethylase (KDM) inhibitor; and
(ii) a pharmaceutical composition comprising a Wnt agonist.

2. The method of claim 1, wherein the administering is to the ear of the subject.

3. The method of claim 2, wherein the administering is to the inner ear of the subject.

4. The method of claim 1, wherein the KDM inhibitor is selected from the group consisting of tranylcypromine (trans-2-phenylcyclopropyl-1-amine, trans-2-PCPA, TCP) and analogs thereof; 2,4-pyridinedicarboxylic acid (2,4-PDCA); 5-Carboxy-8-hydroxyquinoline (IOX1) and n-octyl ester thereof; Pargyline (N-Methyl-N-propargylbenzylamine) or Pargyline hydrochloride (N-Methyl-N-propargylbenzylamine hydrochloride); and C12 ((E)-N′-(1-(5-chloro-2-hydroxyphenyl)ethylidene)-3-(morpholinosulfonyl)benzohydrazide).

5. The method of claim 4, wherein the analog of tranylcypromine is selected from the group consisting of ORY-1001 (rel-N1-[(1R,2S)-2-phenylcyclopropyl]-1,4-cyclohexanediamine, dihydrochloride); S2101 ((1R,2S)-rel-2-[3,5-Difluoro-2-(phenylmethoxy)phenyl]cycloprpanamine hydrochloride); and GSK-LSD1 (rel-N-[(1R,2S)-2-Phenylcyclopropyl]-4-Piperidinamine hydrochloride).

6. The method of claim 1, wherein the Wnt agonist is set forth in Table A.

7. The method of claim 4, wherein the Wnt agonist is set forth in Table A.

8. The method of claim 6, wherein the Wnt agonist is a GSK3β antagonist.

9. The method of claim 7, wherein the Wnt agonist is a GSK3β antagonist.

10. The method of claim 1, wherein the subject is a mammal.

11. The method of claim 10, wherein the subject is a human.

12. The method of claim 11, wherein the subject is at least 3 months of age.

13.-18. (canceled)
